(19) United States
(12) Patent Application Publication
(10) Pub. No.: US 2007/0294789 A1
(43) Pub. Date: Dec. 20, 2007

(54) Photoperiodic control of floret differentiation and yield in plants

(76) Inventors: Hernan Ghiglione, Buenos Aires (AR); Fernanda Gonzalez, Buenos Aires (AR); Charles Chilcott, Research Triangle Park, NC (US); Alfredo Cura, Buenos Aires (AR); Daniel Miralles, Buenos Aires (AR); Tong Zhu, Research Triangle Park, NC (US); Jorge Casal, Buenos Aires (AR)

Correspondence Address: SYNGENTA BIOTECHNOLOGY, INC.;PATENT DEPARTMENT, 3054 CORNWALLIS ROAD, P.O. BOX 12257, RESEARCH TRIANGLE PARK, NC, 27709-2257, US

(21) Appl. No.: 11/729,123

(22) Filed: Mar. 28, 2007

Related U.S. Application Data
(60) Provisional application No. 60/786,436, filed on Mar. 28, 2006.

(51) Int. Cl. A01H 1/00; A01H 5/00

(52) U.S. Cl. 800/294; 800/278; 800/314; 800/320.1; 800/320.3; 800/322

(57) ABSTRACT

A method for improving the yield of a plant is presented. The method uses genetic engineering techniques for transformation of plants to introduce expression cassettes for over- or under-expression of genes involved in photoperiodic control of floret differentiation and degradation. Such methods provide for increased yield at harvest when compared to wild-type plants.

### CLAIM TO PRIOR APPLICATION

This application claims priority to provisional application 60/786,436, filed Mar. 28, 2006.

### FIELD OF THE INVENTION

The present invention relates generally to agricultural biotechnology. More particularly, the invention relates to genes from wheat that are useful in control of the maturation and photoperiodic control of floret development.

### BACKGROUND OF THE INVENTION

Improvement of the agronomic characteristics of crop plants has been ongoing since the beginning of agriculture. Most of the land suitable for crop production is currently being used. As human populations continue to increase, improved crop varieties will be required to adequately provide our food and feed (Trewavas (2001) Plant Physiol. 125: 174-179). To avoid catastrophic famines and malnutrition, future crop cultivars will need to have improved yields with equivalent farm inputs. These cultivars will need to more effectively withstand adverse conditions such as drought, soil salinity or disease, which will be especially important as marginal lands are brought into cultivation. Finally, we will need cultivars with altered nutrient composition to enhance human and animal nutrition, and to enable more efficient food and feed processing, by designing cultivars for specific end-uses. For all these traits, identification of the genes controlling phenotypic expression of traits of interest will be crucial in accelerating development of superior crop germplasm by conventional or transgenic means.

A number of highly-efficient approaches are available to assist identification of genes playing key roles in expression of agronomically-important traits. These include genetics, genomics, bioinformatics, and functional genomics. Genetics is the scientific study of the mechanisms of inheritance. By identifying mutations that alter the pathway or response of interest, classical (or forward) genetics can help to identify the genes involved in these pathways or responses. For example, a mutant with enhanced susceptibility to disease may identify an important component of the plant signal transduction pathway leading from pathogen recognition to disease resistance. Genetics is also the central component in improvement of germplasm by breeding. Through molecular and phenotypic analysis of genetic crosses, loci controlling traits of interest can be mapped and followed in subsequent generations. Knowledge of the genes underlying phenotypic variation between crop accessions can enable development of markers that greatly increase efficiency of the germplasm improvement process, as well as open avenues for discovery of additional superior alleles.

Genomics is the system-level study of an organism's genome, including genes and corresponding gene products—RNA and proteins. At a first level, genomic approaches have provided large datasets of sequence information from diverse plant species, including full-length and partial cDNA sequences, and the complete genomic sequence of a model plant species, Arabidopsis thaliana. Recently, the first draft sequence of a crop plant's genome, that of rice (Oryza sativa), has also become available. Availability of whole genome sequence makes possible the development of tools for system-level study of other molecular complements, such as arrays and chips for use in determining the complement of expressed genes in an organism under specific conditions. Such data can be used as a first indication of the potential for certain genes to play key roles in expression of different plant phenotypes. Bioinformatics approaches interface directly with first-level genomic datasets in allowing for processing to uncover sequences of interest by annotative or other means. Using, for example, similarity searches, alignments and phylogenetic analyses, bioinformatics can often identify homologs of a gene product of interest. Very similar homologs (eg. >˜90% amino acid identity over the entire length of the protein) are very likely orthologs, i.e. share the same function in different organisms.

Functional genomics can be defined as the assignment of function to genes and their products. Functional genomics draws from genetics, genomics and bioinformatics to derive a path toward identifying genes important in a particular pathway or response of interest. Expression analysis, for example, uses high density DNA microarrays (often derived from genomic-scale organismal sequencing) to monitor the mRNA expression of thousands of genes in a single experiment. Experimental treatments can include those eliciting a response of interest, such as the disease resistance response in plants infected with a pathogen. To give additional examples of the use of microarrays, mRNA expression levels can be monitored in distinct tissues over a developmental time course, or in mutants affected in a response of interest. Proteomics can also help to assign function, by assaying the expression and post-translational modifications of hundreds of proteins in a single experiment. Proteomics approaches are in many cases analogous to the approaches taken for monitoring mRNA expression in microarray experiments. Protein-protein interactions can also help to assign proteins to a given pathway or response, by identifying proteins which interact with known components of the pathway or response. For functional genomics, protein-protein interactions are often studied using large-scale yeast two-hybrid assays. Another approach to assigning gene function is to express the corresponding protein in a heterologous host, for example the bacterium Escherichia coli, followed by purification and enzymatic assays.

Ultimately, demonstration of the ability of a gene-of-interest to control a given trait must be derived from experimental testing in plant species of interest. The generation and analysis of plants transgenic for a gene of interest can be used for plant functional genomics, with several advantages. The gene can often be both overexpressed and underexpressed (“knocked out”), thereby increasing the chances of observing a phenotype linking the gene to a pathway or response of interest. Two aspects of transgenic functional genomics help lend a high level of confidence to functional assignment by this approach. First, phenotypic observations are carried out in the context of the living plant. Second, the range of phenotypes observed can be checked and correlated with observed expression levels of the introduced transgene. Transgenic functional genomics is especially valuable in improved cultivar development. Only genes that function in a pathway or response of interest, and that in addition are able to confer a desired trait-based phenotype, are promoted as candidate genes for crop improvement efforts. In some cases, transgenic lines developed for functional genomics studies can be directly utilized in initial stages of product development.

Another approach towards plant functional genomics involves first identifying plant lines with mutations in specific genes of interest, followed by phenotypic evaluation of the consequences of such gene knockouts on the trait under study. Such an approach reveals genes essential for expression of specific traits.

Genes identified through functional genomics can be directly employed in efforts towards germplasm improvement by transgenic means, as described above, or used to develop markers for identification of tracking of alleles-of-interest in mapping and breeding populations. Knowledge of such genes may also enable construction of superior alleles non-existent in nature, by any of a number of molecular methods.

Rapid increases in yield over the last 80 years in row crops have been due in roughly equal measure to improved genetics and improved agronomic practices. In particular, in a crop like maize, the combination of high yielding hybrids and the use of large amounts of nitrogen fertilizer have, under ideal conditions or conditions with irrigation, allowed for yields of greater than 440 bu/acre. Such conditions, however, are not at all ideal or typical, and there is a continuing need for molecular genetic approaches to improvement of yields in plants, particularly crop plants such as maize, soybean, cotton, wheat, canola, sunflower, buckwheat and alfalfa.

Floral structures exhibit extraordinary diversity in Angiosperms. The richness of this natural variation is only beginning to be explored by the combination of genetic, molecular and morphological tools and offers new insight into the evolution of floral development and the understanding of gene function (Buzgo et al., 2004). The list of species under investigation is expanding dramatically, setting the grounds for comparative studies.

Grass species constitute the most important source of food for humankind. Compared to the model eudicot species Arabidopsis thaliana and Antirrhinum majus, the inflorescence of grass plants has two distinctive features. First, the flowers are arranged in small spikes or spickelets, which in turn form the composite inflorescence that may be a spike (e.g. ear in wheat and maize) or a panicle (e.g. rice, terminal tassel in maize). Two bracts or glumes are inserted at the base of each spickelet. Second, the floret contains (outer to inner structures) two leaf-like structures, the lemma (lower position) and the palea (upper position), two lodicules, which occupy the position of the petals, the androecium with three stamens and the gynoecium with two stigmas. Thanks to the developing genetics and genomic tools, rice and maize are emerging as models for grasses and monocots in general (Bommert et al., 2005). Wheat is less suitable for genetic studies and is therefore lagging behind despite its usefulness to investigate the conservation or divergence of mechanisms controlling development, given its evolutionary position closer to rice than maize (Kellogg, 2001) and the different inflorescence morphology. As noted above, the inflorescence is a panicle in rice and a spike in wheat. The rice spickelet meristem gives origin to a single floret, whereas the wheat spickelet meristem is indeterminate and differentiate 6-11 floret primordial but at most 4-5 of these primordia reach the fertile floret stage at anthesis, while the others degenerate and die (Langer and Hanif, 1973; Kirby, 1974). The rice floret contains three additional stamens and the spickelet contains two empty glumes, which are considered to be vestiges of two florets (Bommert et al., 2005).

In wheat, the transition between vegetative and reproductive development is accelerated by long-days (LD). Even if applied after this transition (e.g. when the terminal spickelet primordium is formed at the apex), LD accelerate the rate of floret development (Miralles et al. 2000) and advance the time when the spike achieves its maximum growth rate (Gonzalez et al., 2003; 2005). In addition, LD increase the proportion of florets that interrupt their development and therefore reduce the number of fertile florets per spickelet at the time of anthesis (Miralles et al., 2000; Gonzalez et al., 2003; 2005). The number of fertile florets is a key component of grain yield in wheat. Therefore, searching the genes that change their expression during floret formation and degeneration would be useful in the search for tools to improve yield in this species.

### SUMMARY OF THE INVENTION

The present invention, therefore, relates to a method for improving the yield of a plant. The method uses genetic engineering techniques for transformation of plants to introduce expression cassettes for over- or under-expression of genes involved in photoperiodic control of floret differentiation and degradation. Such methods provide for increased yield at harvest when compared to wild-type plants.

In one embodiment, the invention relates to a method for improving the yield of a plant comprising introducing into the plant an expression cassette capable of altering the expression in the plant of a gene selected from the group consisting of Erecta, ORFx/fw2.2, a squamosa-like gene, an m4 gene, and a BRI1 interactor-like gene, such that their relative level of expression of the gene in long days or short days is increased or decreased in relation to a wild-type plant, so that the overall yield of the plant at harvest is increased. The expression cassette will comprise a promoter capable of driving gene expression in a plant, the gene of interest or an antisense, cosuppressing, or RNAi form of the gene, a terminator, and in alternative embodiments other additional genetic elements such as enhancers, introns, and the like. An expression cassette is introduced using art-recognized techniques such as Agrobacterium- or microparticle bombardment-mediated transformation, or a whiskers-based transformation method. Over-expression or enhanced expression can be achieved using appropriate promoters, enhancers, or techniques such as codon optimization.

### DETAILED DESCRIPTION

For clarity, certain terms used in the specification are defined and presented as follows:

“Associated with/operatively linked” refer to two nucleic acid sequences that are related physically or functionally. For example, a promoter or regulatory DNA sequence is said to be “associated with” a DNA sequence that codes for an RNA or a protein if the two sequences are operatively linked, or situated such that the regulator DNA sequence will affect the expression level of the coding or structural DNA sequence.

A “chimeric construct” is a recombinant nucleic acid sequence in which a promoter or regulatory nucleic acid sequence is operatively linked to, or associated with, a nucleic acid sequence that codes for an mRNA or which is expressed as a protein, such that the regulatory nucleic acid sequence is able to regulate transcription or expression of the associated nucleic acid sequence. The regulatory nucleic acid sequence of the chimeric construct is not normally operatively linked to the associated nucleic acid sequence as found in nature.

A “co-factor” is a natural reactant, such as an organic molecule or a metal ion, required in an enzyme-catalyzed reaction. A co-factor is e.g. NAD(P), riboflavin (including FAD and FMN), folate, molybdopterin, thiamin, biotin, lipoic acid, pantothenic acid and coenzyme A, S-adenosylmethionine, pyridoxal phosphate, ubiquinone, menaquinone. Optionally, a co-factor can be regenerated and reused.

A “coding sequence” is a nucleic acid sequence that is transcribed into RNA such as mRNA, rRNA, tRNA, snRNA, sense RNA or antisense RNA. Preferably the RNA is then translated in an organism to produce a protein.

Complementary: “complementary” refers to two nucleotide sequences that comprise antiparallel nucleotide sequences capable of pairing with one another upon formation of hydrogen bonds between the complementary base residues in the antiparallel nucleotide sequences.

Enzyme activity: means herein the ability of an enzyme to catalyze the conversion of a substrate into a product. A substrate for the enzyme comprises the natural substrate of the enzyme but also comprises analogues of the natural substrate, which can also be converted, by the enzyme into a product or into an analogue of a product. The activity of the enzyme is measured for example by determining the amount of product in the reaction after a certain period of time, or by determining the amount of substrate remaining in the reaction mixture after a certain period of time. The activity of the enzyme is also measured by determining the amount of an unused co-factor of the reaction remaining in the reaction mixture after a certain period of time or by determining the amount of used co-factor in the reaction mixture after a certain period of time. The activity of the enzyme is also measured by determining the amount of a donor of free energy or energy-rich molecule (e.g. ATP, phosphoenolpyruvate, acetyl phosphate or phosphocreatine) remaining in the reaction mixture after a certain period of time or by determining the amount of a used donor of free energy or energy-rich molecule (e.g. ADP, pyruvate, acetate or creatine) in the reaction mixture after a certain period of time.

Expression Cassette: “Expression cassette” as used herein means a nucleic acid molecule capable of directing expression of a particular nucleotide sequence in an appropriate host cell, comprising a promoter operatively linked to the nucleotide sequence of interest which is operatively linked to termination signals. It also typically comprises sequences required for proper translation of the nucleotide sequence. The coding region usually codes for a protein of interest but may also code for a functional RNA of interest, for example antisense RNA or a nontranslated RNA, in the sense or antisense direction. The expression cassette comprising the nucleotide sequence of interest may be chimeric, meaning that at least one of its components is heterologous with respect to at least one of its other components. The expression cassette may also be one that is naturally occurring but has been obtained in a recombinant form useful for heterologous expression. Typically, however, the expression cassette is heterologous with respect to the host, i.e., the particular DNA sequence of the expression cassette does not occur naturally in the host cell and must have been introduced into the host cell or an ancestor of the host cell by a transformation event. The expression of the nucleotide sequence in the expression cassette may be under the control of a constitutive promoter or of an inducible promoter that initiates transcription only when the host cell is exposed to some particular external stimulus. In the case of a multicellular organism, such as a plant, the promoter can also be specific to a particular tissue or organ or stage of development.

Gene: the term “gene” is used broadly to refer to any segment of DNA associated with a biological function. Thus, genes include coding sequences and/or the regulatory sequences required for their expression. Genes also include nonexpressed DNA segments that, for example, form recognition sequences for other proteins. Genes can be obtained from a variety of sources, including cloning from a source of interest or synthesizing from known or predicted sequence information, and may include sequences designed to have desired parameters.

Heterologous/exogenous: The terms “heterologous” and “exogenous” when used herein to refer to a nucleic acid sequence (e.g. a DNA sequence) or a gene, refer to a sequence that originates from a source foreign to the particular host cell or, if from the same source, is modified from its original form. Thus, a heterologous gene in a host cell includes a gene that is endogenous to the particular host cell but has been modified through, for example, the use of DNA shuffling. The terms also include non-naturally occurring multiple copies of a naturally occurring DNA sequence. Thus, the terms refer to a DNA segment that is foreign or heterologous to the cell, or homologous to the cell but in a position within the host cell nucleic acid in which the element is not ordinarily found. Exogenous DNA segments are expressed to yield exogenous polypeptides.

A “homologous” nucleic acid (e.g. DNA) sequence is a nucleic acid (e.g. DNA) sequence naturally associated with a host cell into which it is introduced.

Hybridization: The phrase “hybridizing specifically to” refers to the binding, duplexing, or hybridizing of a molecule only to a particular nucleotide sequence under stringent conditions when that sequence is present in a complex mixture (e.g., total cellular) DNA or RNA. “Bind(s) substantially” refers to complementary hybridization between a probe nucleic acid and a target nucleic acid and embraces minor mismatches that can be accommodated by reducing the stringency of the hybridization media to achieve the desired detection of the target nucleic acid sequence.

Inhibitor: a chemical substance that inactivates the enzymatic activity of a protein such as a biosynthetic enzyme, receptor, signal transduction protein, structural gene product, or transport protein. The term “herbicide” (or “herbicidal compound”) is used herein to define an inhibitor applied to a plant at any stage of development, whereby the herbicide inhibits the growth of the plant or kills the plant.

Interaction: quality or state of mutual action such that the effectiveness or toxicity of one protein or compound on another protein is inhibitory (antagonists) or enhancing (agonists).

A nucleic acid sequence is “isocoding with” a reference nucleic acid sequence when the nucleic acid sequence encodes a polypeptide having the same amino acid sequence as the polypeptide encoded by the reference nucleic acid sequence.

Isogenic: plants that are genetically identical, except that they may differ by the presence or absence of a heterologous DNA sequence.

Isolated: in the context of the present invention, an isolated DNA molecule or an isolated enzyme is a DNA molecule or enzyme that, by the hand of man, exists apart from its native environment and is therefore not a product of nature. An isolated DNA molecule or enzyme may exist in a purified form or may exist in a non-native environment such as, for example, in a transgenic host cell.

Mature protein: protein from which the transit peptide, signal peptide, and/or propeptide portions have been removed.

Minimal Promoter: the smallest piece of a promoter, such as a TATA element, that can support any transcription. A minimal promoter typically has greatly reduced promoter activity in the absence of upstream activation. In the presence of a suitable transcription factor, the minimal promoter functions to permit transcription.

Modified Enzyme Activity: enzyme activity different from that which naturally occurs in a plant (i.e. enzyme activity that occurs naturally in the absence of direct or indirect manipulation of such activity by man), which is tolerant to inhibitors that inhibit the naturally occurring enzyme activity.

Native: refers to a gene that is present in the genome of an untransformed plant cell.

Naturally occurring: the term “naturally occurring” is used to describe an object that can be found in nature as distinct from being artificially produced by man. For example, a protein or nucleotide sequence present in an organism (including a virus), which can be isolated from a source in nature and which has not been intentionally modified by man in the laboratory, is naturally occurring.

Nucleic acid: the term “nucleic acid” refers to deoxyribonucleotides or ribonucleotides and polymers thereof in either single- or double-stranded form. Unless specifically limited, the term encompasses nucleic acids containing known analogues of natural nucleotides which have similar binding properties as the reference nucleic acid and are metabolized in a manner similar to naturally occurring nucleotides. Unless otherwise indicated, a particular nucleic acid sequence also implicitly encompasses conservatively modified variants thereof (e.g. degenerate codon substitutions) and complementary sequences and as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19: 5081 (1991); Ohtsuka et al., J. Biol. Chem. 260: 2605-2608 (1985); Rossolini et al., Mol. Cell. Probes 8: 91-98 (1994)). The terms “nucleic acid” or “nucleic acid sequence” may also be used interchangeably with gene, cDNA, and mRNA encoded by a gene.

“ORF” means open reading frame.

Percent identity: the phrases “percent identical” or “percent identical,” in the context of two nucleic acid or protein sequences, refers to two or more sequences or subsequences that have for example 60%, preferably 70%, more preferably 80%, still more preferably 90%, even more preferably 95%, and most preferably at least 99% nucleotide or amino acid residue identity, when compared and aligned for maximum correspondence, as measured using one of the following sequence comparison algorithms or by visual inspection. Preferably, the percent identity exists over a region of the sequences that is at least about 50 residues in length, more preferably over a region of at least about 100 residues, and most preferably the percent identity exists over at least about 150 residues. In an especially preferred embodiment, the percent identity exists over the entire length of the coding regions.

For sequence comparison, typically one sequence acts as a reference sequence to which test sequences are compared. When using a sequence comparison algorithm, test and reference sequences are input into a computer, subsequence coordinates are designated if necessary, and sequence algorithm program parameters are designated. The sequence comparison algorithm then calculates the percent sequence identity for the test sequence(s) relative to the reference sequence, based on the designated program parameters.

Optimal alignment of sequences for comparison can be conducted, e.g., by the local homology algorithm of Smith & Waterman, Adv. Appl. Math. 2: 482 (1981), by the homology alignment algorithm of Needleman & Wunsch, J. Mol. Biol. 48: 443 (1970), by the search for similarity method of Pearson & Lipman, Proc. Nat'l. Acad. Sci. USA 85: 2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by visual inspection (see generally, Ausubel et al., infra).

One example of an algorithm that is suitable for determining percent sequence identity and sequence similarity is the BLAST algorithm, which is described in Altschul et al., J. Mol. Biol. 215: 403-410 (1990). Software for performing BLAST analyses is publicly available through the National Center for Biotechnology Information (http://www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., 1990). These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when the cumulative alignment score falls off by the quantity X from its maximum achieved value, the cumulative score goes to zero or below due to the accumulation of one or more negative-scoring residue alignments, or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, a cutoff of 100, M=5, N=−4, and a comparison of both strands. For amino acid sequences, the BLASTP program uses as defaults a wordlength (W) of 3, an expectation (E) of 10, and the BLOSUM62 scoring matrix (see Henikoff & Henikoff, Proc. Natl. Acad. Sci. USA 89: 10915 (1989)).

In addition to calculating percent sequence identity, the BLAST algorithm also performs a statistical analysis of the similarity between two sequences (see, e.g., Karlin & Altschul, Proc. Nat'l. Acad. Sci. USA 90: 5873-5787 (1993)). One measure of similarity provided by the BLAST algorithm is the smallest sum probability (P(N)), which provides an indication of the probability by which a match between two nucleotide or amino acid sequences would occur by chance. For example, a test nucleic acid sequence is considered similar to a reference sequence if the smallest sum probability in a comparison of the test nucleic acid sequence to the reference nucleic acid sequence is less than about 0.1, more preferably less than about 0.01, and most preferably less than about 0.001.

Pre-protein: protein that is normally targeted to a cellular organelle, such as a chloroplast, and still comprises its native transit peptide.

Purified: the term “purified,” when applied to a nucleic acid or protein, denotes that the nucleic acid or protein is essentially free of other cellular components with which it is associated in the natural state. It is preferably in a homogeneous state although it can be in either a dry or aqueous solution. Purity and homogeneity are typically determined using analytical chemistry techniques such as polyacrylamide gel electrophoresis or high performance liquid chromatography. A protein that is the predominant species present in a preparation is substantially purified. The term “purified” denotes that a nucleic acid or protein gives rise to essentially one band in an electrophoretic gel. Particularly, it means that the nucleic acid or protein is at least about 50% pure, more preferably at least about 85% pure, and most preferably at least about 99% pure.

Two nucleic acids are “recombined” when sequences from each of the two nucleic acids are combined in a progeny nucleic acid. Two sequences are “directly” recombined when both of the nucleic acids are substrates for recombination. Two sequences are “indirectly recombined” when the sequences are recombined using an intermediate such as a cross-over oligonucleotide. For indirect recombination, no more than one of the sequences is an actual substrate for recombination, and in some cases, neither sequence is a substrate for recombination.

“Regulatory elements” refer to sequences involved in controlling the expression of a nucleotide sequence. Regulatory elements comprise a promoter operatively linked to the nucleotide sequence of interest and termination signals. They also typically encompass sequences required for proper translation of the nucleotide sequence.

Significant Increase: an increase in enzymatic activity that is larger than the margin of error inherent in the measurement technique, preferably an increase by about 2-fold or greater of the activity of the wild-type enzyme in the presence of the inhibitor, more preferably an increase by about 5-fold or greater, and most preferably an increase by about 10-fold or greater.

Significantly less: means that the amount of a product of an enzymatic reaction is reduced by more than the margin of error inherent in the measurement technique, preferably a decrease by about 2-fold or greater of the activity of the wild-type enzyme in the absence of the inhibitor, more preferably an decrease by about 5-fold or greater, and most preferably an decrease by about 10-fold or greater.

Specific Binding/immunological Cross-Reactivity: An indication that two nucleic acid sequences or proteins are substantially identical is that the protein encoded by the first nucleic acid is immunologically cross reactive with, or specifically binds to, the protein encoded by the second nucleic acid. Thus, a protein is typically substantially identical to a second protein, for example, where the two proteins differ only by conservative substitutions. The phrase “specifically (or selectively) binds to an antibody,” or “specifically (or selectively) immunoreactive with,” when referring to a protein or peptide, refers to a binding reaction which is determinative of the presence of the protein in the presence of a heterogeneous population of proteins and other biologics. Thus, under designated immunoassay conditions, the specified antibodies bind to a particular protein and do not bind in a significant amount to other proteins present in the sample. Specific binding to an antibody under such conditions may require an antibody that is selected for its specificity for a particular protein. For example, antibodies raised to the protein with the amino acid sequence encoded by any of the nucleic acid sequences of the invention can be selected to obtain antibodies specifically immunoreactive with that protein and not with other proteins except for polymorphic variants. A variety of immunoassay formats may be used to select antibodies specifically immunoreactive with a particular protein. For example, solid-phase ELISA immunoassays, Western blots, or immunohistochemistry are routinely used to select monoclonal antibodies specifically immunoreactive with a protein. See Harlow and Lane (1988) Antibodies, A Laboratory Manual, Cold Spring Harbor Publications, New York “Harlow and Lane”), for a description of immunoassay formats and conditions that can be used to determine specific immunoreactivity. Typically a specific or selective reaction will be at least twice background signal or noise and more typically more than 10 to 100 times background.

“Stringent hybridization conditions” and “stringent hybridization wash conditions” in the context of nucleic acid hybridization experiments such as Southern and Northern hybridizations are sequence dependent, and are different under different environmental parameters. Longer sequences hybridize specifically at higher temperatures. An extensive guide to the hybridization of nucleic acids is found in Tijssen (1993) Laboratory Techniques in Biochemistry and Molecular Biology-Hybridization with Nucleic Acid Probes part I chapter 2 “Overview of principles of hybridization and the strategy of nucleic acid probe assays” Elsevier, New York. Generally, highly stringent hybridization and wash conditions are selected to be about 5° C. lower than the thermal melting point (Tₘ) for the specific sequence at a defined ionic strength and pH. Typically, under “stringent conditions” a probe will hybridize to its target subsequence, but to no other sequences.

The Tₘ is the temperature (under defined ionic strength and pH) at which 50% of the target sequence hybridizes to a perfectly matched probe. Very stringent conditions are selected to be equal to the Tₘ for a particular probe. An example of stringent hybridization conditions for hybridization of complementary nucleic acids which have more than 100 complementary residues on a filter in a Southern or northern blot is 50% formamide with 1 mg of heparin at 42° C., with the hybridization being carried out overnight. An example of highly stringent wash conditions is 0.15M NaCl at 72° C. for about 15 minutes. An example of stringent wash conditions is a 0.2×SSC wash at 65° C. for 15 minutes (see, Sambrook, infra, for a description of SSC buffer). Often, a high stringency wash is preceded by a low stringency wash to remove background probe signal. An example medium stringency wash for a duplex of, e.g., more than 100 nucleotides, is 1×SSC at 45° C. for 15 minutes. An example low stringency wash for a duplex of, e.g., more than 100 nucleotides, is 4-6×SSC at 40° C. for 15 minutes. For short probes (e.g., about 10 to 50 nucleotides), stringent conditions typically involve salt concentrations of less than about 1.0 M Na ion, typically about 0.01 to 1.0 M Na ion concentration (or other salts) at pH 7.0 to 8.3, and the temperature is typically at least about 30° C. Stringent conditions can also be achieved with the addition of destabilizing agents such as formamide. In general, a signal to noise ratio of 2× (or higher) than that observed for an unrelated probe in the particular hybridization assay indicates detection of a specific hybridization. Nucleic acids that do not hybridize to each other under stringent conditions are still substantially identical if the proteins that they encode are substantially identical. This occurs, e.g., when a copy of a nucleic acid is created using the maximum codon degeneracy permitted by the genetic code.

The following are examples of sets of hybridization/wash conditions that may be used to clone nucleotide sequences that are homologues of reference nucleotide sequences of the present invention: a reference nucleotide sequence preferably hybridizes to the reference nucleotide sequence in 7% sodium dodecyl sulfate (SDS), 0.5 M NaPO₄, 1 mM EDTA at 50° C. with washing in 2×SSC, 0.1% SDS at 50° C., more desirably in 7% sodium dodecyl sulfate (SDS), 0.5 M NaPO₄, 1 mM EDTA at 50° C. with washing in 1×SSC, 0.1% SDS at 50° C., more desirably still in 7% sodium dodecyl sulfate (SDS), 0.5 M NaPO₄, 1 mM EDTA at 50° C. with washing in 0.5×SSC, 0.1% SDS at 50° C., preferably in 7% sodium dodecyl sulfate (SDS), 0.5 M NaPO₄, 1 mM EDTA at 50° C. with washing in 0.1×SSC, 0.1% SDS at 50° C., more preferably in 7% sodium dodecyl sulfate (SDS), 0.5 M NaPO₄, 1 mM EDTA at 50° C. with washing in 0.1×SSC, 0.1% SDS at 65° C.

A “subsequence” refers to a sequence of nucleic acids or amino acids that comprise a part of a longer sequence of nucleic acids or amino acids (e.g., protein) respectively.

Substrate: a substrate is the molecule that an enzyme naturally recognizes and converts to a product in the biochemical pathway in which the enzyme naturally carries out its function, or is a modified version of the molecule, which is also recognized by the enzyme and is converted by the enzyme to a product in an enzymatic reaction similar to the naturally-occurring reaction.

Transformation: a process for introducing heterologous DNA into a plant cell, plant tissue, or plant. Transformed plant cells, plant tissue, or plants are understood to encompass not only the end product of a transformation process, but also transgenic progeny thereof.

“Transformed,” “transgenic,” and “recombinant” refer to a host organism such as a bacterium or a plant into which a heterologous nucleic acid molecule has been introduced. The nucleic acid molecule can be stably integrated into the genome of the host or the nucleic acid molecule can also be present as an extrachromosomal molecule. Such an extrachromosomal molecule can be auto-replicating. Transformed cells, tissues, or plants are understood to encompass not only the end product of a transformation process, but also transgenic progeny thereof. A “non-transformed,” “non-transgenic,” or “non-recombinant” host refers to a wild-type organism, e.g., a bacterium or plant, which does not contain the heterologous nucleic acid molecule.

Viability: “viability” as used herein refers to a fitness parameter of a plant. Plants are assayed for their homozygous performance of plant development, indicating which proteins are essential for plant growth.

### I. General Description of Trait Functional Genomics

The goal of functional genomics is to identify genes controlling expression of organismal phenotypes, and employs a variety of methodologies, including but not limited to bioinformatics, gene expression studies, gene and gene product interactions, genetics, biochemistry and molecular genetics. For example, bioinformatics can assign function to a given gene by identifying genes in heterologous organisms with a high degree of similarity (homology) at the amino acid or nucleotide level. Expression of a gene at the mRNA or protein levels can assign function by linking expression of a gene to an environmental response, a developmental process or a genetic (mutational) or molecular genetic (gene overexpression or underexpression) perturbation. Expression of a gene at the mRNA level can be ascertained either alone (Northern analysis) or in concert with other genes (microarray analysis), whereas expression of a gene at the protein level can be ascertained either alone (native or denatured protein gel or immunoblot analysis) or in concert with other genes (proteomic analysis). Knowledge of protein/protein and protein/DNA interactions can assign function by identifying proteins and nucleic acid sequences acting together in the same biological process. Genetics can assign function to a gene by demonstrating that DNA lesions (mutations) in the gene have a quantifiable effect on the organism, including but not limited to: its development; hormone biosynthesis and response; growth and growth habit (plant architecture); mRNA expression profiles; protein expression profiles; ability to resist diseases; tolerance of abiotic stresses; ability to acquire nutrients; photosynthetic efficiency; altered primary and secondary metabolism; and the composition of various plant organs. Biochemistry can assign function by demonstrating that the protein encoded by the gene, typically when expressed in a heterologous organism, possesses a certain enzymatic activity, alone or in combination with other proteins. Molecular genetics can assign function by overexpressing or underexpressing the gene in the native plant or in heterologous organisms, and observing quantifiable effects as described in functional assignment by genetics above. In functional genomics, any or all of these approaches are utilized, often in concert, to assign genes to functions across any of a number of organismal phenotypes.

It is recognized by those skilled in the art that these different methodologies can each provide data as evidence for the function of a particular gene, and that such evidence is stronger with increasing amounts of data used for functional assignment: preferably from a single methodology, more preferably from two methodologies, and even more preferably from more than two methodologies. In addition, those skilled in the art are aware that different methodologies can differ in the strength of the evidence for the assignment of gene function. Typically, but not always, a datum of biochemical, genetic and molecular genetic evidence is considered stronger than a datum of bioinformatic or gene expression evidence. Finally, those skilled in the art recognize that, for different genes, a single datum from a single methodology can differ in terms of the strength of the evidence provided by each distinct datum for the assignment of the function of these different genes.

The objective of crop trait functional genomics is to identify crop trait genes, i.e. genes capable of conferring useful agronomic traits in crop plants. Such agronomic traits include, but are not limited to: enhanced yield, whether in quantity or quality; enhanced nutrient acquisition and enhanced metabolic efficiency; enhanced or altered nutrient composition of plant tissues used for food, feed, fiber or processing; enhanced utility for agricultural or industrial processing; enhanced resistance to plant diseases; enhanced tolerance of adverse environmental conditions (abiotic stresses) including but not limited to drought, excessive cold, excessive heat, or excessive soil salinity or extreme acidity or alkalinity; and alterations in plant architecture or development, including changes in developmental timing. The deployment of such identified trait genes by either transgenic or non-transgenic means could materially improve crop plants for the benefit of agriculture.

### II. Identifying, Cloning and Sequencing cDNAs

The cloning and sequencing of the cDNAs of the present invention are described in Example 1.

The isolated nucleic acids and proteins of the present invention are usable over a range of plants, monocots and dicots, in particular monocots such as rice, wheat, barley and maize. In a more preferred embodiment, the monocot is a cereal. In a more preferred embodiment, the cereal may be, for example, maize, wheat, barley, oats, rye, millet, sorghum, triticale, secale, einkorn, spelt, emmer, teff, milo, flax, gramma grass, Tripsacum sp., or teosinte. In a most preferred embodiment, the cereal is rice. Other plants genera include, but are not limited to, Cucurbita, Rosa, Vitis, Juglans, Gragaria, Lotus, Medicago, Onobrychis, Trigonella, Vigna, Citrus, Linum, Geranium, Manihot, Daucus, Arabidopsis, Brassica, Raphanus, Sinapis, Atropa, Capsicum, Datura, Hyoscyamus, Lycopersicon, Nicotiana, Solanum, Petunia, Digitalis, Majorana, Ciahorium, Helianthus, Lactuca, Bromus, Asparagus, Antirrhinum, Heterocallis, Nemesis, Pelargonium, Panieum, Pennisetum, Ranunculus, Senecio, Salpiglossis, Cucumis, Browaalia, Glycine, Pisum, Phaseolus, Lolium, Oryza, Avena, Hordeum, Secale, Allium, and Triticum.

The present invention also provides a method of genotyping a plant or plant part comprising a nucleic acid molecule of the present invention. The plant is a monocot or a dicot, such as corn, soybean, cotton, canola, sunflower, buckwheat and alfalfa. Genotyping provides a means of distinguishing homologs of a chromosome pari and can be used to differentiate segregants in a plant population. Molecular marker methods can be used in phylogenetic studies, characterizing genetic relationships among crop varieties, identifying crosses or somatic hybrids, localizing chromosomeal segments affecting mongenic traits, map based cloning, and the study of quantitative inheritance (see Plant Molecular Biology: A Laboratory Manual, Chapter 7, Clark ed., Springer-Verlag, Berlin 1997; Paterson, A. H., “The DNA Revolution”, chapter 2 in Genome Mapping in Plants, Paterson, A. H. ed., Academic Press/R.G. Lands Co., Austin, Tex. 1996).

The method of genotyping may employ any number of molecular marker analytical techniques such as, but not limited to, restriction length polymorphisms (RFLPs). As is well known in the art, RFLPs are produced by differences in the DNA restriction fragment lengths resulting from nucleotide differences between alleles of the same gene. Thus, the present invention provides a method of following segregation of a gene or nucleic acid of the present invention or chromosomal sequences genetically linked by using RFLP analysis. Linked chromosomal sequences are within 50 centiMorgans (50 cM), within 40 or 30 cM, preferably within 20 or 10 cM, more preferably within 5, 3, 2, or 1 cM of the nucleic acid of the invention.

### III. Traits of Interest

The present invention encompasses the identification and isolation of polynucleotides encoding proteins involved in sugar sensing and, ultimately, in nitrogen uptake and carbon metabolism. Altering the expression of genes related to these traits can be used to improve or modify plants and/or grain, as desired. Examples describe the isolated genes of interest and methods of analyzing the alteration of expression and their effects on the plant characteristics.

One aspect of the present invention provides compositions and methods for altering (i.e. increasing or decreasing) the level of nucleic acid molecules and polypeptides of the present invention in plants. In particular, the nucleic acid molecules and polypeptides of the invention are expressed constitutively, temporally or spatially, e.g. at developmental stages, in certain tissues, and/or quantities, which are uncharacteristic of non-recombinantly engineered plants. Therefore, the present invention provides utility in such exemplary applications as altering the specified characteristics identified above.

### VI. Controlling Gene Expression in Transgenic Plants

The invention further relates to transformed cells comprising the nucleic acid molecules, transformed plants, seeds, and plant parts, and methods of modifying phenotypic traits of interest by altering the expression of the genes of the invention.

### A. Modification of Coding Sequences and Adjacent Sequences

The transgenic expression in plants of genes derived from heterologous sources may involve the modification of those genes to achieve and optimize their expression in plants. In particular, bacterial ORFs which encode separate enzymes but which are encoded by the same transcript in the native microbe are best expressed in plants on separate transcripts. To achieve this, each microbial ORF is isolated individually and cloned within a cassette which provides a plant promoter sequence at the 5′ end of the ORF and a plant transcriptional terminator at the 3′ end of the ORF. The isolated ORF sequence preferably includes the initiating ATG codon and the terminating STOP codon but may include additional sequence beyond the initiating ATG and the STOP codon. In addition, the ORF may be truncated, but still retain the required activity; for particularly long ORFs, truncated versions which retain activity may be preferable for expression in transgenic organisms. By “plant promoter” and “plant transcriptional terminator” it is intended to mean promoters and transcriptional terminators that operate within plant cells. This includes promoters and transcription terminators that may be derived from non-plant sources such as viruses (an example is the Cauliflower Mosaic Virus).

In some cases, modification to the ORF coding sequences and adjacent sequence is not required. It is sufficient to isolate a fragment containing the ORF of interest and to insert it downstream of a plant promoter. For example, Gaffney et al. (Science 261: 754-756 (1993)) have expressed the Pseudomonas nahG gene in transgenic plants under the control of the CaMV 35S promoter and the CaMV tml terminator successfully without modification of the coding sequence and with nucleotides of the Pseudomonas gene upstream of the ATG still attached, and nucleotides downstream of the STOP codon still attached to the nahG ORF. Preferably, as little adjacent microbial sequence as possible should be left attached upstream of the ATG and downstream of the STOP codon. In practice, such construction may depend on the availability of restriction sites.

In other cases, the expression of genes derived from microbial sources may provide problems in expression. These problems have been well characterized in the art and are particularly common with genes derived from certain sources such as Bacillus. These problems may apply to the nucleotide sequence of this invention and the modification of these genes can be undertaken using techniques now well known in the art. The following problems may be encountered:

1. Codon Usage.

The preferred codon usage in plants differs from the preferred codon usage in certain microorganisms. Comparison of the usage of codons within a cloned microbial ORF to usage in plant genes (and in particular genes from the target plant) will enable an identification of the codons within the ORF that should preferably be changed. Typically plant evolution has tended towards a strong preference of the nucleotides C and G in the third base position of monocotyledons, whereas dicotyledons often use the nucleotides A or T at this position. By modifying a gene to incorporate preferred codon usage for a particular target transgenic species, many of the problems described below for GC/AT content and illegitimate splicing will be overcome.

2. GC/AT Content.

Plant genes typically have a GC content of more than 35%. ORF sequences which are rich in A and T nucleotides can cause several problems in plants. Firstly, motifs of ATTTA are believed to cause destabilization of messages and are found at the 3′ end of many short-lived mRNAs. Secondly, the occurrence of polyadenylation signals such as AATAAA at inappropriate positions within the message is believed to cause premature truncation of transcription. In addition, monocotyledons may recognize AT-rich sequences as splice sites (see below).

3. Sequences Adjacent to the Initiating Methionine.

Plants differ from microorganisms in that their messages do not possess a defined ribosome-binding site. Rather, it is believed that ribosomes attach to the 5′ end of the message and scan for the first available ATG at which to start translation. Nevertheless, it is believed that there is a preference for certain nucleotides adjacent to the ATG and that expression of microbial genes can be enhanced by the inclusion of a eukaryotic consensus translation initiator at the ATG. Clontech (1993/1994 catalog, page 210, incorporated herein by reference) have suggested one sequence as a consensus translation initiator for the expression of the E. coli uidA gene in plants. Further, Joshi (N.A.R. 15: 6643-6653 (1987), incorporated herein by reference) has compared many plant sequences adjacent to the ATG and suggests another consensus sequence. In situations where difficulties are encountered in the expression of microbial ORFs in plants, inclusion of one of these sequences at the initiating ATG may improve translation. In such cases the last three nucleotides of the consensus may not be appropriate for inclusion in the modified sequence due to their modification of the second AA residue. Preferred sequences adjacent to the initiating methionine may differ between different plant species. A survey of 14 maize genes located in the GenBank database provided the following results:

Position Before the Initiating ATG in 14 Maize Genes:

| | −10 | −9 | −8 | −7 | −6 | −5 | −4 | −3 | −2 | −1 |
|---|---|---|---|---|---|---|---|---|---|---|
| C | 3 | 8 | 4 | 6 | 2 | 5 | 6 | 0 | 10 | 7 |
| T | 3 | 0 | 3 | 4 | 3 | 2 | 1 | 1 | 1 | 0 |
| A | 2 | 3 | 1 | 4 | 3 | 2 | 3 | 7 | 2 | 3 |
| G | 6 | 3 | 6 | 0 | 6 | 5 | 4 | 6 | 1 | 5 |

This analysis can be done for the desired plant species into which the nucleotide sequence is being incorporated, and the sequence adjacent to the ATG modified to incorporate the preferred nucleotides.

4. Removal of Illegitimate Splice Sites.

Genes cloned from non-plant sources and not optimized for expression in plants may also contain motifs which may be recognized in plants as 5′ or 3′ splice sites, and be cleaved, thus generating truncated or deleted messages. These sites can be removed using the techniques well known in the art.

Techniques for the modification of coding sequences and adjacent sequences are well known in the art. In cases where the initial expression of a microbial ORF is low and it is deemed appropriate to make alterations to the sequence as described above, then the construction of synthetic genes can be accomplished according to methods well known in the art. These are, for example, described in the published patent disclosures EP 0 385 962 (to Monsanto), EP 0 359 472 (to Lubrizol) and WO 93/07278 (to Ciba-Geigy), all of which are incorporated herein by reference. In most cases it is preferable to assay the expression of gene constructions using transient assay protocols (which are well known in the art) prior to their transfer to transgenic plants.

### B. Construction of Plant Expression Cassettes

Coding sequences intended for expression in transgenic plants are first assembled in expression cassettes behind a suitable promoter expressible in plants. The expression cassettes may also comprise any further sequences required or selected for the expression of the transgene. Such sequences include, but are not restricted to, transcription terminators, extraneous sequences to enhance expression such as introns, vital sequences, and sequences intended for the targeting of the gene product to specific organelles and cell compartments. These expression cassettes can then be easily transferred to the plant transformation vectors described below. The following is a description of various components of typical expression cassettes.

1. Promoters

The selection of the promoter used in expression cassettes will determine the spatial and temporal expression pattern of the transgene in the transgenic plant. Selected promoters will express transgenes in specific cell types (such as leaf epidermal cells, mesophyll cells, root cortex cells) or in specific tissues or organs (roots, leaves or flowers, for example) and the selection will reflect the desired location of accumulation of the gene product. Alternatively, the selected promoter may drive expression of the gene under various inducing conditions. Promoters vary in their strength, i.e., ability to promote transcription. Depending upon the host cell system utilized, any one of a number of suitable promoters can be used, including the gene's native promoter. The following are non-limiting examples of promoters that may be used in expression cassettes.

a. Constitutive Expression, the Ubiquitin Promoter:

Ubiquitin is a gene product known to accumulate in many cell types and its promoter has been cloned from several species for use in transgenic plants (e.g. sunflower—Binet et al. Plant Science 79: 87-94 (1991); maize—Christensen et al. Plant Molec. Biol. 12: 619-632 (1989); and Arabidopsis—Callis et al., J. Biol. Chem. 265:12486-12493 (1990) and Norris et al., Plant Mol. Biol. 21:895-906 (1993)). The maize ubiquitin promoter has been developed in transgenic monocot systems and its sequence and vectors constructed for monocot transformation are disclosed in the patent publication EP 0 342 926 (to Lubrizol) which is herein incorporated by reference. Taylor et al. (Plant Cell Rep. 12: 491-495 (1993)) describe a vector (pAHC25) that comprises the maize ubiquitin promoter and first intron and its high activity in cell suspensions of numerous monocotyledons when introduced via microprojectile bombardment. The Arabidopsis ubiquitin promoter is ideal for use with the nucleotide sequences of the present invention. The ubiquitin promoter is suitable for gene expression in transgenic plants, both monocotyledons and dicotyledons. Suitable vectors are derivatives of pAHC25 or any of the transformation vectors described in this application, modified by the introduction of the appropriate ubiquitin promoter and/or intron sequences.

b. Constitutive Expression, the CaMV 35S Promoter:

Construction of the plasmid pCGN1761 is described in the published patent application EP 0 392 225 (Example 23), which is hereby incorporated by reference. pCGN1761 contains the “double” CaMV 35S promoter and the tml transcriptional terminator with a unique EcoRI site between the promoter and the terminator and has a pUC-type backbone. A derivative of pCGN1761 is constructed which has a modified polylinker which includes NotI and XhoI sites in addition to the existing EcoRI site. This derivative is designated pCGN1761ENX. pCGN1761ENX is useful for the cloning of cDNA sequences or coding sequences (including microbial ORF sequences) within its polylinker for the purpose of their expression under the control of the 35S promoter in transgenic plants. The entire 35S promoter-coding sequence-tml terminator cassette of such a construction can be excised by HindIII, SphI, SalI, and XbaI sites 5′ to the promoter and XbaI, BamHI and BglI sites 3′ to the terminator for transfer to transformation vectors such as those described below. Furthermore, the double 35S promoter fragment can be removed by 5′ excision with HindIII, SphI, SalI, XbaI, or PstI, and 3′ excision with any of the polylinker restriction sites (EcoRI, NotI or XhoI) for replacement with another promoter. If desired, modifications around the cloning sites can be made by the introduction of sequences that may enhance translation. This is particularly useful when overexpression is desired. For example, pCGN1761ENX may be modified by optimization of the translational initiation site as described in Example 37 of U.S. Pat. No. 5,639,949, incorporated herein by reference.

c. Constitutive Expression, the Actin Promoter:

Several isoforms of actin are known to be expressed in most cell types and consequently the actin promoter is a good choice for a constitutive promoter. In particular, the promoter from the rice ActI gene has been cloned and characterized (McElroy et al. Plant Cell 2: 163-171 (1990)). A 1.3 kb fragment of the promoter was found to contain all the regulatory elements required for expression in rice protoplasts. Furthermore, numerous expression vectors based on the ActI promoter have been constructed specifically for use in monocotyledons (McElroy et al. Mol. Gen. Genet. 231: 150-160 (1991)). These incorporate the ActI-intron 1, AdhI 5′ flanking sequence and AdhI-intron 1 (from the maize alcohol dehydrogenase gene) and sequence from the CaMV 35S promoter. Vectors showing highest expression were fusions of 35S and ActI intron or the ActI 5′ flanking sequence and the ActI intron. Optimization of sequences around the initiating ATG (of the GUS reporter gene) also enhanced expression. The promoter expression cassettes described by McElroy et al. (Mol. Gen. Genet. 231: 150-160 (1991)) can be easily modified for gene expression and are particularly suitable for use in monocotyledonous hosts. For example, promoter-containing fragments is removed from the McElroy constructions and used to replace the double 35S promoter in pCGN1761ENX, which is then available for the insertion of specific gene sequences. The fusion genes thus constructed can then be transferred to appropriate transformation vectors. In a separate report, the rice ActI promoter with its first intron has also been found to direct high expression in cultured barley cells (Chibbar et al. Plant Cell Rep. 12: 506-509 (1993)).

d. Inducible Expression, PR-1 Promoters:

The double 35S promoter in pCGN1761ENX may be replaced with any other promoter of choice that will result in suitably high expression levels. By way of example, one of the chemically regulatable promoters described in U.S. Pat. No. 5,614,395, such as the tobacco PR-1a promoter, may replace the double 35S promoter. Alternately, the Arabidopsis PR-1 promoter described in Lebel et al., Plant J. 16:223-233 (1998) may be used. The promoter of choice is preferably excised from its source by restriction enzymes, but can alternatively be PCR-amplified using primers that carry appropriate terminal restriction sites. Should PCR-amplification be undertaken, the promoter should be re-sequenced to check for amplification errors after the cloning of the amplified promoter in the target vector. The chemically/pathogen regulatable tobacco PR-1a promoter is cleaved from plasmid pCIB1004 (for construction, see example 21 of EP 0 332 104, which is hereby incorporated by reference) and transferred to plasmid pCGN1761ENX (Uknes et al., Plant Cell 4: 645-656 (1992)). pCIB1004 is cleaved with NcoI and the resultant 3′ overhang of the linearized fragment is rendered blunt by treatment with T4 DNA polymerase. The fragment is then cleaved with HindIII and the resultant PR-1a promoter-containing fragment is gel purified and cloned into pCGN1761ENX from which the double 35S promoter has been removed. This is accomplished by cleavage with XhoI and blunting with T4 polymerase, followed by cleavage with Hind III, and isolation of the larger vector-terminator containing fragment into which the pCIB1004 promoter fragment is cloned. This generates a pCGN1761ENX derivative with the PR-1a promoter and the tml terminator and an intervening polylinker with unique EcoRI and NotI sites. The selected coding sequence can be inserted into this vector, and the fusion products (i.e. promoter-gene-terminator) can subsequently be transferred to any selected transformation vector, including those described infra. Various chemical regulators may be employed to induce expression of the selected coding sequence in the plants transformed according to the present invention, including the benzothiadiazole, isonicotinic acid, and salicylic acid compounds disclosed in U.S. Pat. Nos. 5,523,311 and 5,614,395.

e. Inducible Expression, an Ethanol-Inducible Promoter:

A promoter inducible by certain alcohols or ketones, such as ethanol, may also be used to confer inducible expression of a coding sequence of the present invention. Such a promoter is for example the alcA gene promoter from Aspergillus nidulans (Caddick et al. (1998) Nat. Biotechnol 16:177-180). In A. nidulans, the alcA gene encodes alcohol dehydrogenase I, the expression of which is regulated by the AlcR transcription factors in presence of the chemical inducer. For the purposes of the present invention, the CAT coding sequences in plasmid palcA:CAT comprising a alcA gene promoter sequence fused to a minimal 35S promoter (Caddick et al. (1998) Nat. Biotechnol 16:177-180) are replaced by a coding sequence of the present invention to form an expression cassette having the coding sequence under the control of the alcA gene promoter. This is carried out using methods well known in the art.

f. Inducible Expression, a Glucocorticoid-Inducible Promoter:

Induction of expression of a nucleic acid sequence of the present invention using systems based on steroid hormones is also contemplated. For example, a glucocorticoid-mediated induction system is used (Aoyama and Chua (1997) The Plant Journal 11: 605-612) and gene expression is induced by application of a glucocorticoid, for example a synthetic glucocorticoid, preferably dexamethasone, preferably at a concentration ranging from 0.1 mM to 1 mM, more preferably from 10 mM to 100 mM. For the purposes of the present invention, the luciferase gene sequences are replaced by a nucleic acid sequence of the invention to form an expression cassette having a nucleic acid sequence of the invention under the control of six copies of the GAL4 upstream activating sequences fused to the 35S minimal promoter. This is carried out using methods well known in the art. The trans-acting factor comprises the GAL4 DNA-binding domain (Keegan et al. (1986). Science 231: 699-704) fused to the transactivating domain of the herpes viral protein VP16 (Triezenberg et al. (1988) Genes Devel. 2: 718-729) fused to the hormone-binding domain of the rat glucocorticoid receptor (Picard et al. (1988) Cell 54: 1073-1080). The expression of the fusion protein is controlled either by a promoter known in the art or described here. This expression cassette is also comprised in the plant comprising a nucleic acid sequence of the invention fused to the 6×GAL4/minimal promoter. Thus, tissue- or organ-specificity of the fusion protein is achieved leading to inducible tissue- or organ-specificity of the insecticidal toxin.

g. Root Specific Expression:

Another pattern of gene expression is root expression. A suitable root promoter is the promoter of the maize metallothionein-like (MTL) gene described by de Framond (FEBS 290: 103-106 (1991)) and also in U.S. Pat. No. 5,466,785, incorporated herein by reference. This “MTL” promoter is transferred to a suitable vector such as pCGN1761ENX for the insertion of a selected gene and subsequent transfer of the entire promoter-gene-terminator cassette to a transformation vector of interest.

h. Wound-Inducible Promoters:

Wound-inducible promoters may also be suitable for gene expression. Numerous such promoters have been described (e.g. Xu et al. Plant Molec. Biol. 22: 573-588 (1993), Logemann et al. Plant Cell 1: 151-158 (1989), Rohrmeier & Lehle, Plant Molec. Biol. 22: 783-792 (1993), Firek et al. Plant Molec. Biol. 22: 129-142 (1993), Warner et al. Plant J. 3: 191-201 (1993)) and all are suitable for use with the instant invention. Logemann et al. describe the 5′ upstream sequences of the dicotyledonous potato wunI gene. Xu et al. show that a wound-inducible promoter from the dicotyledon potato (pin2) is active in the monocotyledon rice. Further, Rohrmeier & Lehle describe the cloning of the maize WipI cDNA which is wound induced and which can be used to isolate the cognate promoter using standard techniques. Similar, Firek et al. and Warner et al. have described a wound-induced gene from the monocotyledon Asparagus officinalis, which is expressed at local wound and pathogen invasion sites. Using cloning techniques well known in the art, these promoters can be transferred to suitable vectors, fused to the genes pertaining to this invention, and used to express these genes at the sites of plant wounding.

i. Pith-Preferred Expression:

Patent Application WO 93/07278, which is herein incorporated by reference, describes the isolation of the maize trpA gene, which is preferentially expressed in pith cells. The gene sequence and promoter extending up to −1726 bp from the start of transcription are presented. Using standard molecular biological techniques, this promoter, or parts thereof, can be transferred to a vector such as pCGN1761 where it can replace the 35S promoter and be used to drive the expression of a foreign gene in a pith-preferred manner. In fact, fragments containing the pith-preferred promoter or parts thereof can be transferred to any vector and modified for utility in transgenic plants.

j. Leaf-Specific Expression:

A maize gene encoding phosphoenol carboxylase (PEPC) has been described by Hudspeth & Grula (Plant Molec Biol 12: 579-589 (1989)). Using standard molecular biological techniques the promoter for this gene can be used to drive the expression of any gene in a leaf-specific manner in transgenic plants.

k. Pollen-Specific Expression:

WO 93/07278 describes the isolation of the maize calcium-dependent protein kinase (CDPK) gene which is expressed in pollen cells. The gene sequence and promoter extend up to 1400 bp from the start of transcription. Using standard molecular biological techniques, this promoter or parts thereof, can be transferred to a vector such as pCGN1761 where it can replace the 35S promoter and be used to drive the expression of a nucleic acid sequence of the invention in a pollen-specific manner.

2. Transcriptional Terminators

A variety of transcriptional terminators are available for use in expression cassettes. These are responsible for the termination of transcription beyond the transgene and correct mRNA polyadenylation. Appropriate transcriptional terminators are those that are known to function in plants and include the CaMV 35S terminator, the tml terminator, the nopaline synthase terminator and the pea rbcS E9 terminator. These can be used in both monocotyledons and dicotyledons. In addition, a gene's native transcription terminator may be used.

3. Sequences for the Enhancement or Regulation of Expression

Numerous sequences have been found to enhance gene expression from within the transcriptional unit and these sequences can be used in conjunction with the genes of this invention to increase their expression in transgenic plants.

Various intron sequences have been shown to enhance expression, particularly in monocotyledonous cells. For example, the introns of the maize AdhI gene have been found to significantly enhance the expression of the wild-type gene under its cognate promoter when introduced into maize cells. Intron 1 was found to be particularly effective and enhanced expression in fusion constructs with the chloramphenicol acetyltransferase gene (Callis et al., Genes Develop. 1: 1183-1200 (1987)). In the same experimental system, the intron from the maize bronze1 gene had a similar effect in enhancing expression. Intron sequences have been routinely incorporated into plant transformation vectors, typically within the non-translated leader.

A number of non-translated leader sequences derived from viruses are also known to enhance expression, and these are particularly effective in dicotyledonous cells. Specifically, leader sequences from Tobacco Mosaic Virus (TMV, the “W-sequence”), Maize Chlorotic Mottle Virus (MCMV), and Alfalfa Mosaic Virus (AMV) have been shown to be effective in enhancing expression (e.g. Gallie et al. Nucl. Acids Res. 15: 8693-8711 (1987); Skuzeski et al. Plant Molec. Biol. 15: 65-79 (1990)). Other leader sequences known in the art include but are not limited to: picornavirus leaders, for example, EMCV leader (Encephalomyocarditis 5′ noncoding region) (Elroy-Stein, O., Fuerst, T. R., and Moss, B. PNAS USA 86:6126-6130 (1989)); potyvirus leaders, for example, TEV leader (Tobacco Etch Virus) (Allison et al., 1986); MDMV leader (Maize Dwarf Mosaic Virus); Virology 154:9-20); human immunoglobulin heavy-chain binding protein (BiP) leader, (Macejak, D. G., and Sarnow, P., Nature 353: 90-94 (1991); untranslated leader from the coat protein mRNA of alfalfa mosaic virus (AMV RNA 4), (Jobling, S. A., and Gehrke, L., Nature 325:622-625 (1987); tobacco mosaic virus leader (TMV), (Gallie, D. R. et al., Molecular Biology of RNA, pages 237-256 (1989); and Maize Chlorotic Mottle Virus leader (MCMV) (Lommel, S. A. et al., Virology 81:382-385 (1991). See also, Della-Cioppa et al., Plant Physiology 84:965-968 (1987).

In addition to incorporating one or more of the aforementioned elements into the 5′ regulatory region of a target expression cassette of the invention, other elements peculiar to the target expression cassette may also be incorporated. Such elements include but are not limited to a minimal promoter. By minimal promoter it is intended that the basal promoter elements are inactive or nearly so without upstream activation. Such a promoter has low background activity in plants when there is no transactivator present or when enhancer or response element binding sites are absent. One minimal promoter that is particularly useful for target genes in plants is the Bz1 minimal promoter, which is obtained from the bronze1 gene of maize. The Bz1 core promoter is obtained from the “myc” mutant Bz1-luciferase construct pBz1LucR98 via cleavage at the NheI site located at −53 to −58. Roth et al., Plant Cell 3: 317 (1991). The derived Bz1 core promoter fragment thus extends from −53 to +227 and includes the Bz1 intron-1 in the 5′ untranslated region. Also useful for the invention is a minimal promoter created by use of a synthetic TATA element. The TATA element allows recognition of the promoter by RNA polymerase factors and confers a basal level of gene expression in the absence of activation (see generally, Mukumoto (1993) Plant Mol Biol 23: 995-1003; Green (2000) Trends Biochem Sci 25: 59-63).

### C. Construction of Plant Transformation Vectors

Numerous transformation vectors available for plant transformation are known to those of ordinary skill in the plant transformation arts, and the genes pertinent to this invention can be used in conjunction with any such vectors. The selection of vector will depend upon the preferred transformation technique and the target species for transformation. For certain target species, different antibiotic or herbicide selection markers may be preferred. Selection markers used routinely in transformation include the nptII gene, which confers resistance to kanamycin and related antibiotics (Messing & Vierra. Gene 19: 259-268 (1982); Bevan et al., Nature 304:184-187 (1983)), the bar gene, which confers resistance to the herbicide phosphinothricin (White et al., Nucl. Acids Res 18: 1062 (1990), Spencer et al. Theor. Appl. Genet. 79: 625-631 (1990)), the hph gene, which confers resistance to the antibiotic hygromycin (Blochinger & Diggelmann, Mol Cell Biol 4: 2929-2931), and the dhfr gene, which confers resistance to methatrexate (Bourouis et al., EMBO J. 2(7): 1099-1104 (1983)), the EPSPS gene, which confers resistance to glyphosate (U.S. Pat. Nos. 4,940,935 and 5,188,642), and the mannose-6-phosphate isomerase gene, which provides the ability to metabolize mannose (U.S. Pat. Nos. 5,767,378 and 5,994,629).

1. Vectors Suitable for Agrobacterium Transformation

Many vectors are available for transformation using Agrobacterium tumefaciens. These typically carry at least one T-DNA border sequence and include vectors such as pBIN19 (Bevan, Nucl. Acids Res. (1984)). Below, the construction of two typical vectors suitable for Agrobacterium transformation is described.

a. pCIB200 and pCIB2001:

The binary vectors pCIB200 and pCIB2001 are used for the construction of recombinant vectors for use with Agrobacterium and are constructed in the following manner. pTJS75kan is created by NarI digestion of pTJS75 (Schmidhauser & Helinski, J. Bacteriol. 164: 446-455 (1985)) allowing excision of the tetracycline-resistance gene, followed by insertion of an AccI fragment from pUC4K carrying an NPTII (Messing & Vierra, Gene 19: 259-268 (1982): Bevan et al., Nature 304: 184-187 (1983): McBride et al., Plant Molecular Biology 14: 266-276 (1990)). XhoI linkers are ligated to the EcoRV fragment of PCIB7 which contains the left and right T-DNA borders, a plant selectable nos/nptII chimeric gene and the pUC polylinker (Rothstein et al., Gene 53: 153-161 (1987)), and the XhoI-digested fragment are cloned into SalI-digested pTJS75kan to create pCIB200 (see also EP 0 332 104, example 19). pCIB200 contains the following unique polylinker restriction sites: EcoRI, SstI, KpnI, BglII, XbaI, and SalI. pCIB2001 is a derivative of pCIB200 created by the insertion into the polylinker of additional restriction sites. Unique restriction sites in the polylinker of pCIB2001 are EcoRI, SstI, KpnI, BglII, XbaI, SalI, MluI, BclI, AvrII, ApaI, HpaI, and StuI. pCIB2001, in addition to containing these unique restriction sites also has plant and bacterial kanamycin selection, left and right T-DNA borders for Agrobacterium-mediated transformation, the RK2-derived trfA function for mobilization between E. coli and other hosts, and the OriT and OriV functions also from RK2. The pCIB2001 polylinker is suitable for the cloning of plant expression cassettes containing their own regulatory signals.

b. pCIB10 and Hygromycin Selection Derivatives Thereof:

The binary vector pCIB10 contains a gene encoding kanamycin resistance for selection in plants and T-DNA right and left border sequences and incorporates sequences from the wide host-range plasmid pRK252 allowing it to replicate in both E. coli and Agrobacterium. Its construction is described by Rothstein et al. (Gene 53: 153-161 (1987)). Various derivatives of pCIB10 are constructed which incorporate the gene for hygromycin B phosphotransferase described by Gritz et al. (Gene 25: 179-188 (1983)). These derivatives enable selection of transgenic plant cells on hygromycin only (pCIB743), or hygromycin and kanamycin (pCIB715, pCIB717).

2. Vectors Suitable for Non-Agrobacterium Transformation

Transformation without the use of Agrobacterium tumefaciens circumvents the requirement for T-DNA sequences in the chosen transformation vector and consequently vectors lacking these sequences can be utilized in addition to vectors such as the ones described above which contain T-DNA sequences. Transformation techniques that do not rely on Agrobacterium include transformation via particle bombardment, protoplast uptake (e.g. PEG and electroporation) and microinjection. The choice of vector depends largely on the preferred selection for the species being transformed. Below, the construction of typical vectors suitable for non-Agrobacterium transformation is described.

a. pCIB3064:

pCIB3064 is a pUC-derived vector suitable for direct gene transfer techniques in combination with selection by the herbicide basta (or phosphinothricin). The plasmid pCIB246 comprises the CaMV 35S promoter in operational fusion to the E. coli GUS gene and the CaMV 35S transcriptional terminator and is described in the PCT published application WO 93/07278. The 35S promoter of this vector contains two ATG sequences 5′ of the start site. These sites are mutated using standard PCR techniques in such a way as to remove the ATGs and generate the restriction sites SspI and PvuII. The new restriction sites are 96 and 37 bp away from the unique SalI site and 101 and 42 bp away from the actual start site. The resultant derivative of pCIB246 is designated pCIB3025. The GUS gene is then excised from pCIB3025 by digestion with SalI and SacI, the termini rendered blunt and religated to generate plasmid pCIB3060. The plasmid pJIT82 is obtained from the John Innes Centre, Norwich and the a 400 bp SmaI fragment containing the bar gene from Streptomyces viridochromogenes is excised and inserted into the HpaI site of pCIB3060 (Thompson et al. EMBO J. 6: 2519-2523 (1987)). This generated pCIB3064, which comprises the bar gene under the control of the CaMV 35S promoter and terminator for herbicide selection, a gene for ampicillin resistance (for selection in E. coli) and a polylinker with the unique sites SphI, PstI, HindIII, and BamHI. This vector is suitable for the cloning of plant expression cassettes containing their own regulatory signals.

b. pSOG19 and pSOG35:

pSOG35 is a transformation vector that utilizes the E. coli gene dihydrofolate reductase (DFR) as a selectable marker conferring resistance to methotrexate. PCR is used to amplify the 35S promoter (−800 bp), intron 6 from the maize Adh1 gene (−550 bp) and 18 bp of the GUS untranslated leader sequence from pSOG10. A 250-bp fragment encoding the E. coli dihydrofolate reductase type II gene is also amplified by PCR and these two PCR fragments are assembled with a SacI-PstI fragment from pB1221 (Clontech) which comprises the pUC19 vector backbone and the nopaline synthase terminator. Assembly of these fragments generates pSOG19 which contains the 35S promoter in fusion with the intron 6 sequence, the GUS leader, the DHFR gene and the nopaline synthase terminator. Replacement of the GUS leader in pSOG19 with the leader sequence from Maize Chlorotic Mottle Virus (MCMV) generates the vector pSOG35. pSOG19 and pSOG35 carry the pUC gene for ampicillin resistance and have HindIII, SphI, PstI and EcoRI sites available for the cloning of foreign substances.

3. Vector Suitable for Chloroplast Transformation

For expression of a nucleotide sequence of the present invention in plant plastids, plastid transformation vector pPH143 (WO 97/32011, example 36) is used. The nucleotide sequence is inserted into pPH143 thereby replacing the PROTOX coding sequence. This vector is then used for plastid transformation and selection of transformants for spectinomycin resistance. Alternatively, the nucleotide sequence is inserted in pPH143 so that it replaces the aadH gene. In this case, transformants are selected for resistance to PROTOX inhibitors.

### D. Transformation

Once a nucleic acid sequence of the invention has been cloned into an expression system, it is transformed into a plant cell. The receptor and target expression cassettes of the present invention can be introduced into the plant cell in a number of art-recognized ways. Methods for regeneration of plants are also well known in the art. For example, Ti plasmid vectors have been utilized for the delivery of foreign DNA, as well as direct DNA uptake, liposomes, electroporation, microinjection, and microprojectiles. In addition, bacteria from the genus Agrobacterium can be utilized to transform plant cells. Below are descriptions of representative techniques for transforming both dicotyledonous and monocotyledonous plants, as well as a representative plastid transformation technique.

1. Transformation of Dicotyledons

Transformation techniques for dicotyledons are well known in the art and include Agrobacterium-based techniques and techniques that do not require Agrobacterium. Non-Agrobacterium techniques involve the uptake of exogenous genetic material directly by protoplasts or cells. This can be accomplished by PEG or electroporation mediated uptake, particle bombardment-mediated delivery, or microinjection. Examples of these techniques are described by Paszkowski et al., EMBO J. 3: 2717-2722 (1984), Potrykus et al., Mol. Gen. Genet. 199: 169-177 (1985), Reich et al., Biotechnology 4: 1001-1004 (1986), and Klein et al., Nature 327: 70-73 (1987). In each case the transformed cells are regenerated to whole plants using standard techniques known in the art.

Agrobacterium-mediated transformation is a preferred technique for transformation of dicotyledons because of its high efficiency of transformation and its broad utility with many different species. Agrobacterium transformation typically involves the transfer of the binary vector carrying the foreign DNA of interest (e.g. pCIB200 or pCIB2001) to an appropriate Agrobacterium strain which may depend of the complement of vir genes carried by the host Agrobacterium strain either on a co-resident Ti plasmid or chromosomally (e.g. strain CIB542 for pCIB200 and pCIB2001 (Uknes et al. Plant Cell 5: 159-169 (1993)). The transfer of the recombinant binary vector to Agrobacterium is accomplished by a triparental mating procedure using E. coli carrying the recombinant binary vector, a helper E. coli strain which carries a plasmid such as pRK2013 and which is able to mobilize the recombinant binary vector to the target Agrobacterium strain. Alternatively, the recombinant binary vector can be transferred to Agrobacterium by DNA transformation (Hofgen & Willmitzer, Nucl. Acids Res. 16: 9877 (1988)).

Transformation of the target plant species by recombinant Agrobacterium usually involves co-cultivation of the Agrobacterium with explants from the plant and follows protocols well known in the art. Transformed tissue is regenerated on selectable medium carrying the antibiotic or herbicide resistance marker present between the binary plasmid T-DNA borders.

Another approach to transforming plant cells with a gene involves propelling inert or biologically active particles at plant tissues and cells. This technique is disclosed in U.S. Pat. Nos. 4,945,050, 5,036,006, and 5,100,792 all to Sanford et al. Generally, this procedure involves propelling inert or biologically active particles at the cells under conditions effective to penetrate the outer surface of the cell and afford incorporation within the interior thereof. When inert particles are utilized, the vector can be introduced into the cell by coating the particles with the vector containing the desired gene. Alternatively, the target cell can be surrounded by the vector so that the vector is carried into the cell by the wake of the particle. Biologically active particles (e.g., dried yeast cells, dried bacterium or a bacteriophage, each containing DNA sought to be introduced) can also be propelled into plant cell tissue.

2. Transformation of Monocotyledons

Transformation of most monocotyledon species has now also become routine. Preferred techniques include direct gene transfer into protoplasts using PEG or electroporation techniques, and particle bombardment into callus tissue. Transformations can be undertaken with a single DNA species or multiple DNA species (i.e. co-transformation) and both these techniques are suitable for use with this invention. Co-transformation may have the advantage of avoiding complete vector construction and of generating transgenic plants with unlinked loci for the gene of interest and the selectable marker, enabling the removal of the selectable marker in subsequent generations, should this be regarded desirable. However, a disadvantage of the use of co-transformation is the less than 100% frequency with which separate DNA species are integrated into the genome (Schocher et al. Biotechnology 4: 1093-1096 (1986)).

Patent Applications EP 0 292 435, EP 0 392 225, and WO 93/07278 describe techniques for the preparation of callus and protoplasts from an elite inbred line of maize, transformation of protoplasts using PEG or electroporation, and the regeneration of maize plants from transformed protoplasts. Gordon-Kamm et al. (Plant Cell 2: 603-618 (1990)) and Fromm et al. (Biotechnology 8: 833-839 (1990)) have published techniques for transformation of A188-derived maize line using particle bombardment. Furthermore, WO 93/07278 and Koziel et al. (Biotechnology 11: 194-200 (1993)) describe techniques for the transformation of elite inbred lines of maize by particle bombardment. This technique utilizes immature maize embryos of 1.5-2.5 mm length excised from a maize ear 14-15 days after pollination and a PDS-100He Biolistics device for bombardment.

Transformation of rice can also be undertaken by direct gene transfer techniques utilizing protoplasts or particle bombardment. Protoplast-mediated transformation has been described for Japonica-types and Indica-types (Zhang et al. Plant Cell Rep Z: 379-384 (1988); Shimamoto et al. Nature 338: 274-277 (1989); Datta et al. Biotechnology 8: 736-740 (1990)). Both types are also routinely transformable using particle bombardment (Christou et al. Biotechnology 9: 957-962 (1991)). Furthermore, WO 93/21335 describes techniques for the transformation of rice via electroporation.

Patent Application EP 0 332 581 describes techniques for the generation, transformation and regeneration of Pooideae protoplasts. These techniques allow the transformation of Dactylis and wheat. Furthermore, wheat transformation has been described by Vasil et al. (Biotechnology 10: 667-674 (1992)) using particle bombardment into cells of type C long-term regenerable callus, and also by Vasil et al. (Biotechnology 11: 1553-1558 (1993)) and Weeks et al. (Plant Physiol. 102: 1077-1084 (1993)) using particle bombardment of immature embryos and immature embryo-derived callus. A preferred technique for wheat transformation, however, involves the transformation of wheat by particle bombardment of immature embryos and includes either a high sucrose or a high maltose step prior to gene delivery. Prior to bombardment, any number of embryos (0.75-1 mm in length) are plated onto MS medium with 3% sucrose (Murashiga & Skoog, Physiologia Plantarum 15: 473-497 (1962)) and 3 mg/l 2,4-D for induction of somatic embryos, which is allowed to proceed in the dark. On the chosen day of bombardment, embryos are removed from the induction medium and placed onto the osmoticum (i.e. induction medium with sucrose or maltose added at the desired concentration, typically 15%). The embryos are allowed to plasmolyze for 2-3 hours and are then bombarded. Twenty embryos per target plate is typical, although not critical. An appropriate gene-carrying plasmid (such as pCIB3064 or pSG35) is precipitated onto micrometer size gold particles using standard procedures. Each plate of embryos is shot with the DuPont Biolistics® helium device using a burst pressure of ˜1000 psi using a standard 80 mesh screen. After bombardment, the embryos are placed back into the dark to recover for about 24 hours (still on osmoticum). After 24 hrs, the embryos are removed from the osmoticum and placed back onto induction medium where they stay for about a month before regeneration. Approximately one month later the embryo explants with developing embryogenic callus are transferred to regeneration medium (MS+1 mg/liter NAA, 5 mg/liter GA), further containing the appropriate selection agent (10 mg/l basta in the case of pCIB3064 and 2 mg/l methotrexate in the case of pSOG35). After approximately one month, developed shoots are transferred to larger sterile containers known as “GA7s” which contain half-strength MS, 2% sucrose, and the same concentration of selection agent.

Transformation of monocotyledons using Agrobacterium has also been described. See, WO 94/00977 and U.S. Pat. No. 5,591,616, both of which are incorporated herein by reference. See also, Negrotto et al., Plant Cell Reports 19: 798-803 (2000), incorporated herein by reference.

For this example, rice (Oryza sativa) is used for generating transgenic plants. Various rice cultivars can be used (Hiei et al., 1994, Plant Journal 6:271-282; Dong et al., 1996, Molecular Breeding 2:267-276; Hiei et al., 1997, Plant Molecular Biology, 35:205-218). Also, the various media constituents described below may be either varied in quantity or substituted. Embryogenic responses are initiated and/or cultures are established from mature embryos by culturing on MS-CIM medium (MS basal salts, 4.3 g/liter; B5 vitamins (200×), 5 ml/liter; Sucrose, 30 g/liter; proline, 500 mg/liter; glutamine, 500 mg/liter; casein hydrolysate, 300 mg/liter; 2,4-D (1 mg/ml), 2 ml/liter; adjust pH to 5.8 with 1 N KOH; Phytagel, 3 g/liter). Either mature embryos at the initial stages of culture response or established culture lines are inoculated and co-cultivated with the Agrobacterium tumefaciens strain LBA4404 (Agrobacterium) containing the desired vector construction. Agrobacterium is cultured from glycerol stocks on solid YPC medium (100 mg/L spectinomycin and any other appropriate antibiotic) for ˜2 days at 28° C. Agrobacterium is re-suspended in liquid MS-CIM medium. The Agrobacterium culture is diluted to an OD600 of 0.2-0.3 and acetosyringone is added to a final concentration of 200 uM. Acetosyringone is added before mixing the solution with the rice cultures to induce Agrobacterium for DNA transfer to the plant cells. For inoculation, the plant cultures are immersed in the bacterial suspension. The liquid bacterial suspension is removed and the inoculated cultures are placed on co-cultivation medium and incubated at 22° C. for two days. The cultures are then transferred to MS-CIM medium with Ticarcillin (400 mg/liter) to inhibit the growth of Agrobacterium. For constructs utilizing the PMI selectable marker gene (Reed et al., In Vitro Cell. Dev. Biol.-Plant 37:127-132), cultures are transferred to selection medium containing Mannose as a carbohydrate source (MS with 2% Mannose, 300 mg/liter Ticarcillin) after 7 days, and cultured for 3-4 weeks in the dark. Resistant colonies are then transferred to regeneration induction medium (MS with no 2,4-D, 0.5 mg/liter IAA, 1 mg/liter zeatin, 200 mg/liter timentin 2% Mannose and 3% Sorbitol) and grown in the dark for 14 days. Proliferating colonies are then transferred to another round of regeneration induction media and moved to the light growth room. Regenerated shoots are transferred to GA7 containers with GA7-1 medium (MS with no hormones and 2% Sorbitol) for 2 weeks and then moved to the greenhouse when they are large enough and have adequate roots. Plants are transplanted to soil in the greenhouse (T₀ generation) grown to maturity, and the T₁ seed is harvested.

3. Transformation of Plastids

Seeds of Nicotiana tabacum c.v. ‘Xanthi nc’ are germinated seven per plate in a 1″ circular array on T agar medium and bombarded 12-14 days after sowing with 1 μm tungsten particles (M10, Biorad, Hercules, Calif.) coated with DNA from plasmids pPH143 and pPH145 essentially as described (Svab, Z. and Maliga, P. (1993) PNAS 90, 913-917). Bombarded seedlings are incubated on T medium for two days after which leaves are excised and placed abaxial side up in bright light (350-500 μmol photons/m²/s) on plates of RMOP medium (Svab, Z., Hajdukiewicz, P. and Maliga, P. (1990) PNAS 87, 8526-8530) containing 500 μg/ml spectinomycin dihydrochloride (Sigma, St. Louis, Mo.). Resistant shoots appearing underneath the bleached leaves three to eight weeks after bombardment are subcloned onto the same selective medium, allowed to form callus, and secondary shoots isolated and subcloned. Complete segregation of transformed plastid genome copies (homoplasmicity) in independent subclones is assessed by standard techniques of Southern blotting (Sambrook et al., (1989) Molecular Cloning: A Laboratory Manual, Cold Spring Harbor Laboratory, Cold Spring Harbor). BamHI/EcoRI-digested total cellular DNA (Mettler, I. J. (1987) Plant Mol Biol Reporter 5, 346-349) is separated on 1% Tris-borate (TBE) agarose gels, transferred to nylon membranes (Amersham) and probed with ³²P-labeled random primed DNA sequences corresponding to a 0.7 kb BamHI/HindIII DNA fragment from pC8 containing a portion of the rps7/12 plastid targeting sequence. Homoplasmic shoots are rooted aseptically on spectinomycin-containing MS/IBA medium (McBride, K. E. et al. (1994) PNAS 91, 7301-7305) and transferred to the greenhouse.

### V. Breeding and Seed Production

### A. Breeding

The plants obtained via transformation with a nucleic acid sequence of the present invention can be any of a wide variety of plant species, including those of monocots and dicots; however, the plants used in the method of the invention are preferably selected from the list of agronomically important target crops set forth supra. The expression of a gene of the present invention in combination with other characteristics important for production and quality can be incorporated into plant lines through breeding. Breeding approaches and techniques are known in the art. See, for example, Welsh J. R., Fundamentals of Plant Genetics and Breeding, John Wiley & Sons, NY (1981); Crop Breeding, Wood D. R. (Ed.) American Society of Agronomy Madison, Wis. (1983); Mayo O., The Theory of Plant Breeding, Second Edition, Clarendon Press, Oxford (1987); Singh, D. P., Breeding for Resistance to Diseases and Insect Pests, Springer-Verlag, NY (1986); and Wricke and Weber, Quantitative Genetics and Selection Plant Breeding, Walter de Gruyter and Co., Berlin (1986).

The genetic properties engineered into the transgenic seeds and plants described above are passed on by sexual reproduction or vegetative growth and can thus be maintained and propagated in progeny plants. Generally said maintenance and propagation make use of known agricultural methods developed to fit specific purposes such as tilling, sowing or harvesting. Specialized processes such as hydroponics or greenhouse technologies can also be applied. As the growing crop is vulnerable to attack and damages caused by insects or infections as well as to competition by weed plants, measures are undertaken to control weeds, plant diseases, insects, nematodes, and other adverse conditions to improve yield. These include mechanical measures such a tillage of the soil or removal of weeds and infected plants, as well as the application of agrochemicals such as herbicides, fungicides, gametocides, nematicides, growth regulants, ripening agents and insecticides.

Use of the advantageous genetic properties of the transgenic plants and seeds according to the invention can further be made in plant breeding, which aims at the development of plants with improved properties such as tolerance of pests, herbicides, or stress, improved nutritional value, increased yield, or improved structure causing less loss from lodging or shattering. The various breeding steps are characterized by well-defined human intervention such as selecting the lines to be crossed, directing pollination of the parental lines, or selecting appropriate progeny plants. Depending on the desired properties, different breeding measures are taken. The relevant techniques are well known in the art and include but are not limited to hybridization, inbreeding, backcross breeding, multiline breeding, variety blend, interspecific hybridization, aneuploid techniques, etc. Hybridization techniques also include the sterilization of plants to yield male or female sterile plants by mechanical, chemical, or biochemical means. Cross pollination of a male sterile plant with pollen of a different line assures that the genome of the male sterile but female fertile plant will uniformly obtain properties of both parental lines. Thus, the transgenic seeds and plants according to the invention can be used for the breeding of improved plant lines, that for example, increase the effectiveness of conventional methods such as herbicide or pesticide treatment or allow one to dispense with said methods due to their modified genetic properties. Alternatively new crops with improved stress tolerance can be obtained, which, due to their optimized genetic “equipment”, yield harvested product of better quality than products that were not able to tolerate comparable adverse developmental conditions.

### B. Seed Production

In seed production, germination quality and uniformity of seeds are essential product characteristics. As it is difficult to keep a crop free from other crop and weed seeds, to control seedborne diseases, and to produce seed with good germination, fairly extensive and well-defined seed production practices have been developed by seed producers, who are experienced in the art of growing, conditioning and marketing of pure seed. Thus, it is common practice for the farmer to buy certified seed meeting specific quality standards instead of using seed harvested from his own crop. Propagation material to be used as seeds is customarily treated with a protectant coating comprising herbicides, insecticides, fungicides, bactericides, nematicides, molluscicides, or mixtures thereof. Customarily used protectant coatings comprise compounds such as captan, carboxin, thiram (TMTD®), methalaxyl (Apron®), and pirimiphos-methyl (Actellic®). If desired, these compounds are formulated together with further carriers, surfactants or application-promoting adjuvants customarily employed in the art of formulation to provide protection against damage caused by bacterial, fungal or animal pests. The protectant coatings may be applied by impregnating propagation material with a liquid formulation or by coating with a combined wet or dry formulation. Other methods of application are also possible such as treatment directed at the buds or the fruit.

### VI. Alteration of Expression of Nucleic Acid Molecules

The alteration in expression of the nucleic acid molecules of the present invention is achieved in one of the following ways:

A. “Sense” Suppression

Alteration of the expression of a nucleotide sequence of the present invention, preferably reduction of its expression, is obtained by “sense” suppression (referenced in e.g. Jorgensen et al. (1996) Plant Mol. Biol. 31, 957-973). In this case, the entirety or a portion of a nucleotide sequence of the present invention is comprised in a DNA molecule. The DNA molecule is preferably operatively linked to a promoter functional in a cell comprising the target gene, preferably a plant cell, and introduced into the cell, in which the nucleotide sequence is expressible. The nucleotide sequence is inserted in the DNA molecule in the “sense orientation”, meaning that the coding strand of the nucleotide sequence can be transcribed. In a preferred embodiment, the nucleotide sequence is fully translatable and all the genetic information comprised in the nucleotide sequence, or portion thereof, is translated into a polypeptide. In another preferred embodiment, the nucleotide sequence is partially translatable and a short peptide is translated. In a preferred embodiment, this is achieved by inserting at least one premature stop codon in the nucleotide sequence, which bring translation to a halt. In another more preferred embodiment, the nucleotide sequence is transcribed but no translation product is being made. This is usually achieved by removing the start codon, e.g. the “ATG”, of the polypeptide encoded by the nucleotide sequence. In a further preferred embodiment, the DNA molecule comprising the nucleotide sequence, or a portion thereof, is stably integrated in the genome of the plant cell. In another preferred embodiment, the DNA molecule comprising the nucleotide sequence, or a portion thereof, is comprised in an extrachromosomally replicating molecule.

In transgenic plants containing one of the DNA molecules described immediately above, the expression of the nucleotide sequence corresponding to the nucleotide sequence comprised in the DNA molecule is preferably reduced. Preferably, the nucleotide sequence in the DNA molecule is at least 70% identical to the nucleotide sequence the expression of which is reduced, more preferably it is at least 80% identical, yet more preferably at least 90% identical, yet more preferably at least 95% identical, yet more preferably at least 99% identical.

B. “Anti-Sense” Suppression

In another preferred embodiment, the alteration of the expression of a nucleotide sequence of the present invention, preferably the reduction of its expression is obtained by “anti-sense” suppression. The entirety or a portion of a nucleotide sequence of the present invention is comprised in a DNA molecule. The DNA molecule is preferably operatively linked to a promoter functional in a plant cell, and introduced in a plant cell, in which the nucleotide sequence is expressible. The nucleotide sequence is inserted in the DNA molecule in the “anti-sense orientation”, meaning that the reverse complement (also called sometimes non-coding strand) of the nucleotide sequence can be transcribed. In a preferred embodiment, the DNA molecule comprising the nucleotide sequence, or a portion thereof, is stably integrated in the genome of the plant cell. In another preferred embodiment the DNA molecule comprising the nucleotide sequence, or a portion thereof, is comprised in an extrachromosomally replicating molecule. Several publications describing this approach are cited for further illustration (Green, P. J. et al., Ann. Rev. Biochem. 55:569-597 (1986); van der Krol, A. R. et al, Antisense Nuc. Acids & Proteins, pp. 125-141 (1991); Abel, P. P. et al., PNASroc. Natl. Acad. Sci. USA 86:6949-6952 (1989); Ecker, J. R. et al., Proc. Natl. Acad. Sci. USANAS 83:5372-5376 (Aug. 1986)).

In transgenic plants containing one of the DNA molecules described immediately above, the expression of the nucleotide sequence corresponding to the nucleotide sequence comprised in the DNA molecule is preferably reduced. Preferably, the nucleotide sequence in the DNA molecule is at least 70% identical to the nucleotide sequence the expression of which is reduced, more preferably it is at least 80% identical, yet more preferably at least 90% identical, yet more preferably at least 95% identical, yet more preferably at least 99% identical.

C. Homologous Recombination

In another preferred embodiment, at least one genomic copy corresponding to a nucleotide sequence of the present invention is modified in the genome of the plant by homologous recombination as further illustrated in Paszkowski et al., EMBO Journal 7:4021-26 (1988). This technique uses the property of homologous sequences to recognize each other and to exchange nucleotide sequences between each by a process known in the art as homologous recombination. Homologous recombination can occur between the chromosomal copy of a nucleotide sequence in a cell and an incoming copy of the nucleotide sequence introduced in the cell by transformation. Specific modifications are thus accurately introduced in the chromosomal copy of the nucleotide sequence. In one embodiment, the regulatory elements of the nucleotide sequence of the present invention are modified. Such regulatory elements are easily obtainable by screening a genomic library using the nucleotide sequence of the present invention, or a portion thereof, as a probe. The existing regulatory elements are replaced by different regulatory elements, thus altering expression of the nucleotide sequence, or they are mutated or deleted, thus abolishing the expression of the nucleotide sequence. In another embodiment, the nucleotide sequence is modified by deletion of a part of the nucleotide sequence or the entire nucleotide sequence, or by mutation. Expression of a mutated polypeptide in a plant cell is also contemplated in the present invention. More recent refinements of this technique to disrupt endogenous plant genes have been described (Kempin et al., Nature 389:802-803 (1997) and Miao and Lam, Plant J., 7:359-365 (1995).

In another preferred embodiment, a mutation in the chromosomal copy of a nucleotide sequence is introduced by transforming a cell with a chimeric oligonucleotide composed of a contiguous stretch of RNA and DNA residues in a duplex conformation with double hairpin caps on the ends. An additional feature of the oligonucleotide is for example the presence of 2′-O-methylation at the RNA residues. The RNA/DNA sequence is designed to align with the sequence of a chromosomal copy of a nucleotide sequence of the present invention and to contain the desired nucleotide change. For example, this technique is further illustrated in U.S. Pat. No. 5,501,967 and Zhu et al. (1999) Proc. Natl. Acad. Sci. USA 96: 8768-8773.

D. Zinc Finger Proteins

A zinc finger protein that binds a nucleotide sequence of the present invention or to its regulatory region is also used to alter expression of the nucleotide sequence. Preferably, transcription of the nucleotide sequence is reduced or increased. Zinc finger proteins are for example described in Beerli et al. (1998) PNAS 95:14628-14633., or in WO 95/19431, WO 98/54311, or WO 96/06166, all incorporated herein by reference in their entirety.

E. dsRNA

Alteration of the expression of a nucleotide sequence of the present invention is also obtained by dsRNA interference as described for example in WO 99/32619, WO 99/53050 or WO 99/61631, all incorporated herein by reference in their entirety. In another preferred embodiment, the alteration of the expression of a nucleotide sequence of the present invention, preferably the reduction of its expression, is obtained by double-stranded RNA (dsRNA) interference. The entirety or, preferably a portion of a nucleotide sequence of the present invention is comprised in a DNA molecule. The size of the DNA molecule is preferably from 100 to 1000 nucleotides or more; the optimal size to be determined empirically. Two copies of the identical DNA molecule are linked, separated by a spacer DNA molecule, such that the first and second copies are in opposite orientations. In the preferred embodiment, the first copy of the DNA molecule is in the reverse complement (also known as the non-coding strand) and the second copy is the coding strand; in the most preferred embodiment, the first copy is the coding strand, and the second copy is the reverse complement. The size of the spacer DNA molecule is preferably 200 to 10,000 nucleotides, more preferably 400 to 5000 nucleotides and most preferably 600 to 1500 nucleotides in length. The spacer is preferably a random piece of DNA, more preferably a random piece of DNA without homology to the target organism for dsRNA interference, and most preferably a functional intron which is effectively spliced by the target organism. The two copies of the DNA molecule separated by the spacer are operatively linked to a promoter functional in a plant cell, and introduced in a plant cell, in which the nucleotide sequence is expressible. In a preferred embodiment, the DNA molecule comprising the nucleotide sequence, or a portion thereof, is stably integrated in the genome of the plant cell. In another preferred embodiment the DNA molecule comprising the nucleotide sequence, or a portion thereof, is comprised in an extrachromosomally replicating molecule. Several publications describing this approach are cited for further illustration (Waterhouse et al. (1998) PNAS 95:13959-13964; Chuang and Meyerowitz (2000) PNAS 97:4985-4990; Smith et al. (2000) Nature 407:319-320). Alteration of the expression of a nucleotide sequence by dsRNA interference is also described in, for example WO 99/32619, WO 99/53050 or WO 99/61631, all incorporated herein by reference in their entirety

In transgenic plants containing one of the DNA molecules described immediately above, the expression of the nucleotide sequence corresponding to the nucleotide sequence comprised in the DNA molecule is preferably reduced. Preferably, the nucleotide sequence in the DNA molecule is at least 70% identical to the nucleotide sequence the expression of which is reduced, more preferably it is at least 80% identical, yet more preferably at least 90% identical, yet more preferably at least 95% identical, yet more preferably at least 99% identical.

F. Insertion of a DNA Molecule (Insertional Mutagenesis)

In another preferred embodiment, a DNA molecule is inserted into a chromosomal copy of a nucleotide sequence of the present invention, or into a regulatory region thereof. Preferably, such DNA molecule comprises a transposable element capable of transposition in a plant cell, such as e.g. Ac/Ds, Em/Spm, mutator. Alternatively, the DNA molecule comprises a T-DNA border of an Agrobacterium T-DNA. The DNA molecule may also comprise a recombinase or integrase recognition site which can be used to remove part of the DNA molecule from the chromosome of the plant cell. Methods of insertional mutagenesis using T-DNA, transposons, oligonucleotides or other methods known to those skilled in the art are also encompassed. Methods of using T-DNA and transposon for insertional mutagenesis are described in Winkler et al. (1989) Methods Mol. Biol. 82:129-136 and Martienssen (1998) PNAS 95:2021-2026, incorporated herein by reference in their entireties.

G. Deletion Mutagenesis

In yet another embodiment, a mutation of a nucleic acid molecule of the present invention is created in the genomic copy of the sequence in the cell or plant by deletion of a portion of the nucleotide sequence or regulator sequence. Methods of deletion mutagenesis are known to those skilled in the art. See, for example, Miao et al, (1995) Plant J. 7:359.

In yet another embodiment, this deletion is created at random in a large population of plants by chemical mutagenesis or irradiation and a plant with a deletion in a gene of the present invention is isolated by forward or reverse genetics. Irradiation with fast neutrons or gamma rays is known to cause deletion mutations in plants (Silverstone et al, (1998) Plant Cell, 10:155-169; Bruggemann et al., (1996) Plant J., 10:755-760; Redei and Koncz in Methods in Arabidopsis Research, World Scientific Press (1992), pp. 16-82). Deletion mutations in a gene of the present invention can be recovered in a reverse genetics strategy using PCR with pooled sets of genomic DNAs as has been shown in C. elegans (Liu et al., (1999), Genome Research, 9:859-867.). A forward genetics strategy would involve mutagenesis of a line displaying PTGS followed by screening the M2 progeny for the absence of PTGS. Among these mutants would be expected to be some that disrupt a gene of the present invention. This could be assessed by Southern blot or PCR for a gene of the present invention with genomic DNA from these mutants.

H. Over-Expression in a Plant Cell

In yet another preferred embodiment, a nucleotide sequence of the present invention encoding a polypeptide is over-expressed. Examples of nucleic acid molecules and expression cassettes for over-expression of a nucleic acid molecule of the present invention are described above. Methods known to those skilled in the art of over-expression of nucleic acid molecules are also encompassed by the present invention.

In a preferred embodiment, the expression of the nucleotide sequence of the present invention is altered in every cell of a plant. This is for example obtained though homologous recombination or by insertion in the chromosome. This is also for example obtained by expressing a sense or antisense RNA, zinc finger protein or ribozyme under the control of a promoter capable of expressing the sense or antisense RNA, zinc finger protein or ribozyme in every cell of a plant. Constitutive expression, inducible, tissue-specific or developmentally-regulated expression are also within the scope of the present invention and result in a constitutive, inducible, tissue-specific or developmentally-regulated alteration of the expression of a nucleotide sequence of the present invention in the plant cell. Constructs for expression of the sense or antisense RNA, zinc finger protein or ribozyme, or for over-expression of a nucleotide sequence of the present invention, are prepared and transformed into a plant cell according to the teachings of the present invention, e.g. as described herein.

Standard recombinant DNA and molecular cloning techniques used here are well known in the art and are described by J. Sambrook, et al., Molecular Cloning: A Laboratory Manual, 3d Ed., Cold Spring Harbor, N.Y.: Cold Spring Harbor Laboratory Press (2001); by T. J. Silhavy, M. L. Berman, and L. W. Enquist, Experiments with Gene Fusions, Cold Spring Harbor Laboratory, Cold Spring Harbor, N.Y. (1984) and by Ausubel, F. M. et al., Current Protocols in Molecular Biology, New York, John Wiley and Sons Inc., (1988), Reiter, et al., Methods in Arabidopsis Research, World Scientific Press (1992), and Schultz et al., Plant Molecular Biology Manual, Kluwer Academic Publishers (1998).

The examples set forth herein are given by way of illustration and explanation, and are not intended to be limiting in any way.

### EXAMPLE 1

### Spike Growth and Development

Plants of wheat (Triticum aestivum L) were sown in the field under natural SD. When the plants had reached the terminal spickelet stage of reproductive development, half of the plots were switched to LD conditions, which consisted of a natural photoperiod extended with 6 h of low-fluence rate white light. Plant samples were periodically harvested between terminal spickelet and anthesis, i.e. the developmental window when floral development takes place inside the spickelets. Time course data were plotted against thermal time (that measures time as the sum of the daily difference between average temperatures and the threshold temperature) rather than chronological time because thermal time attenuates the impact of temperature fluctuations and make data more comparable for different years or locations. The linear phase of spike growth was anticipated under LD compared to SD. Under LD anthesis took place at 450° C.d, when SD plants were at the boot stage where only the awns are visible 30-50 mm outside the sheath of the uppermost leaf. Under SD anthesis occurred at 550° C.d. LD, compared to SD, reduced spike weight at anthesis (i.e. before the transition to grain filling).

Floret Development

LD also accelerated floret development scored with the developmental scale of Waddington et al. (1983). LD accelerated the development of both the carpels and the anthers.

The number of fertile florets was reduced under LD compared to SD. In wheat, spickelets are indeterminate and differentiate 6-11 floret primordial but at most 4-5 of these primordia reach the fertile floret stage at anthesis, while the others degenerate and die (Langer and Hanif, 1973; Kirby, 1974). In one instance, a floret from a central spickelet interrupted the progression of its development before reaching score 6 on the scale of Waddington et al. (1983) under either SD or LD. Another floret, however, reached the fertile stage in SD-treated plants but it failed to do so under LD. This occurred despite the initially faster development under LD. Floret degeneration can start after reaching a variable range of developmental stages, including Waddington scores 6 or 7. Before abortion the floral organs showed progressive de-differentiation and shrinking.

Transcriptome Patterns During Floret Formation and Abortion

To explore the molecular basis of the developmental progression that takes place between terminal spickelet and anthesis, we harvested the main ear of plants grown under SD or LD in the field in two different years. Samples were hybridised to Affymetrix custom wheat microarrays containing 38564 genes. Normalised data were log-transformed and fitted to regression models including log harvest time (linear or quadratic relationships) and either light treatment (SD or LD) or log harvest time×light treatment as independent variables. The 8308 genes showing significant regression (q<0.05) in the first of the two independent experiments were grouped in 6 clusters containing at least 200 genes. Three of these clusters showed average expression values that either increased (clusters 1, 5) or decreased (cluster 2) with time and showed relatively weak effects of daylength. For these clusters, the small difference between SD and LD was largely eliminated by plotting expression values against the score of a central spickelet floret on the Waddington scale (data not shown). In other words, these genes showed similar levels of expression in SD and LD spikes compared at equivalent developmental status. Conversely, the other three clusters showed larger differences as LD either increased (clusters 3, 4) or decreased (clusters 6) average expression relative to SD. Genes involved in cellular metabolism dominate clusters 1, 5 and 3; mainly carbohydrate metabolism genes (all three clusters) and either photosynthesis-related genes (cluster 1) or lipid metabolism genes (clusters 3 and 5). Ribosomal protein genes (cellular metabolism), and genes involved in transcription and RNA processing dominate Cluster 2. Cluster 4 includes a relatively large proportion of transcription factors and cluster 6 genes involved in signal transduction, particularly protein kinases. The specific genes mentioned in subsequent paragraphs were found significant (q<0.05) in each one of the two field experiments analysed independently.

Getting Ready for Light: Carbon-Metabolism, Photosynthesis, Photo-Protection and Photomorphogenic Genes Increase their Expression Before the Emergence of the Ear

The development of the ear occurs largely inside the tube formed by the sheaths of the leaves. Before emergence from this leaf cover, the ear is exposed to relatively poor levels of photosynthetic radiation and low red/far-red ratios. Although grain filling starts after anthesis, pre-anthesis carbohydrates are known to contribute to the final pool of reserves (Gebbing and Schnyder, 1999). A large proportion of the genes of clusters 1 and 5, which show increasing expression, are related to carbohydrate metabolism. These genes include Glyceraldehyde 3-phosphate dehydrogenase, Phosphoglycerate kinase; glucose-6-phosphate dehydrogenase and Sucrose Synthase 1 and 2 (cluster 1), etc.

The content of chlorophyll also increased during ear development (data not shown) and clusters 1 and 5 show a large proportion of genes that encode proteins involved in light harvesting and photochemical reactions of photosynthesis. The list includes Photosystem I antenna proteins, Photosystem II core complex proteins, chlorophyll a/b binding protein, different subunits to Photosystem I reaction center and Thylakoid lumenal 25.6 kDa proteins in cluster 1. Carotenoids are important as accessory antenna pigments and for their role against oxidative stress. Enzymes involved in carotenoid/xanthophylls synthesis, such as phytoene synthase, lycopene cyclase, beta-carotene hydroxylase, and zeta-carotene desaturase are present in cluster 1. The photosynthetic capacity of the ear contributes to grain filling after anthesis and this photosynthetic capacity builds up while the ear is enclosed in the sheath tube.

Several genes of enzymes involved in the metabolism of flavonoids, which exert a protective function against excess irradiation are part of cluster 1. These enzymes include phenylalanine ammonia-lyase, cinnamate-4-hydroxylase, chalcone isomerase, 4-coumaroyl:CoA-ligase, dihydroflavonol 4-reductase, Isoflavone reductase, leucoanthocyanidin dioxygenase, and O-methyltransferase.

Several genes involved in the perception and response to the light signals increased their expression during floret development (cluster 1 and 5). They include two photoreceptor-related genes, HO1, a heme oxygenase that participates in the phytochrome chromophore biosynthesis (Davis et al., 2001) and PHOTOTROPIN (formerly, NPH1, Non Phototropic Hypocotyls 1), which encode a UV-A/blue-light photoreceptor. CONSTANS and its target Hd3a, a rice ortholog of the Arabidopsis FT gene, two genes involved in the photoperiodic control of flowering in Arabidopsis and rice, also showed increasing expression levels. While positive players in light signalling increase their expression, COP9 a repressor of photo-morphogenesis (Serino and Deng, 2003) showed decreasing expression. COP-INTERACTING PROTEIN 7 increased its expression during floret development.

Genes with Predicted Function in the Development of Floret Organs Increase their Expression During Floret Formation

The development of florets occurs between terminal spickelet and anthesis, and many genes, homologs to those involved in the development of floral organs in other species showed increasing expression during this period (Cluster 1). The list includes several pollen-related genes such as MALE STERILITY 2 (MS2), a pollen-specific pectinesterase, which in Arabidopsis is involved in the formation of pollen wall (Aarts et al., 1997); PIP1, an acuaporin differentially expressed during anther and stigma development in tobacco (Bots et al., 2005); CER1 and CUT1/CER6, which participate in the elimination to the very long chain lipids from the pollen coats (Fiebig et al., 2000).

Two MADS box genes with likely function in floret development increased their expression between terminal spikelet and anthesis stages (cluster 1). OsMADS8 (Lee et al., 2003; Kang et al., 1997) and HvAG1. Other genes include ACE (Adhesion of Calyx Edges), originally identified as a mutation that causes floral organs to fuse together and reduced fertility most likely as because of fusions that pistil emergence (Lolle et al., 1998); EREBP (Ethylene Responsive Element Binding Protein) a class of transcription factors involved in the specification of floral organ identity, establishment of floral meristem identity, suppression of floral meristem indeterminancy, and development of the ovule and seed coat (Ohto et al., 2005); EFA27, a rice abscisic acid-induced protein whose barley homolog is expressed during kernel development (Jang et al., 2003); and LOX2 a lipoxygenase from barley also expressed in developing grains (Van Mechelen et al., 1999).

Genes with Predicted Function in Cell Proliferation Show Decreasing Expression Between Terminal Spickelet and Anthesis

Cluster 2 characterized by a gradual decrease, contained several genes involved in cell proliferation and/or meristem development. Since in many organisms proliferation and programmed cell death are controlled by signalling elements with opposite effects on both processes, it is tempting to associate genes of this group with the interruption of floret development. Cluster 2 includes REVOLUTA and ATHB-8, two members of class III homeodomain-leucine zipper gene family of transcription factors, which share overlapping and antagonistic functions (Prigge et al., 2005). In particular, the revoluta mutant of Arabidopsis forms abortive structures in the inflorescence (Prigge et al., 2005; Talbert et al., 1995). AGO1, present in cluster 2, is a member of the multigene ARGONAUTE family (Kidner and Martienssen, 2005) involved in stem cell function and organ polarity, as well as the closely related protein PINHEAD (Lynn et al., 1999). ago1 mutants produce reduced number of flowers amongst their most conspicuous phenotype features (Morel et al., 2002). In Arabidopsis, TSO1 is a floral-specific regulator of cell division (Liu et al., 1997), a nuclear protein highly expressed in developing ovules and microspores (Hauser et al., 2000; Sato et al., 2001). The tso1 mutation blocks the formation of floral organs in whorls 2, 3 and 4 (Liu et al., 1997). A KNOX1 (knotted-like homeobox 1) gene and the distantly related HOX1 gene (Bellmann and Werr, 1992), are transcription factors also included in cluster 2. KNOX1 genes are involved in the maintenance of shoot apical meristem, determination of cell fate and differentiation of vegetative tissues and have previously been reported in young wheat spikes (Takumi et al., 2000; Sano et al., 2005).

APK1, is a protein kinase that interacts with the flowering homeotic protein AGAMOUS (Conner and Liu, 2000; Ito et al., 1997). FEN1 is an enzyme involved in DNA replication and repair, expressed strongly in proliferating tissues such as root tips and young leaves. In rice, mRNA from FEN1 is abundant in the shoot apical meristem, tiller bud, leaf primordia, ligule primordia and marginal meristem of young leaves. This suggest that FEN1, is expressed in tissues rich in proliferating cells, and its expression, may be required for cell growth and organ formation (Kimura et al., 2000). PESCADILLO-like proteins, which play a role in ribosome biogenesis, cell size and cell proliferation in animals system (Prisco et al., 2004). ASY1, which was reported in Arabidopsis as essential for homologous chromosome synapses and initially detected in pollen mother cells during meiotic interphase, as numerous punctate foci distributed over the chromatin (Armstrong et al., 2002). In rice, suppression to PAIR2, an ortolog of ASY1, results in a defective meiosis suggesting the important role to ASY1 in this process (Nonomura et al., 2004).

Several histone genes (H1, H2A and B, H3 and H4) are present in cluster 2. H1 is an abundant component of eukaryotic chromatin that is thought to stabilize higher-order chromatin structures. In Arabidopsis, reduction of H1 expression, resulted in an aberrant developmental phenotype and delayed flowering (Wierzbicki and Jerzmanowski, 2005). In transgenic tobacco, increasing H1A and H1B expressions (in detriment to H1C and H1D), caused aberrations in flower development and male sterility (Prymakowska-Bosak et al., 1999). Transcrips from H2A are found in root tips from tomato and pea, expressing cells were concentrated near the apex, and their distribution was consistent with that expected of cycling cells (Koning et al., 1991). Other H2A transcripts were found in non-dividing cortical cells that are known to undergo endoduplication during the late maturation phase of primary development (Koning et al., 1991). Jeong et al. (2003) reported changes in the expression of several histone genes, including H2A and B, in response to a variety of stress signals. H3 shows expression in developmental tissues with differences in male or female meiosis in mouse (López-Fernandez et al., 1997).

Cluster 2 also includes several transcription-related genes, such as KAP2 a protein proposed to be involved in control of DNA recombination and transcription (Lindsay et al., 2002), E2F and E2F-like (E2L3) two transcription factors involved in cell proliferation and cell cycle control in Arabidopsis (Kosugi and Ohashi 2002), SOF1 that in yeast is a nucleolar protein and is essential for cell grown and a component of the RNA processing machinery (Jansen et al., 1993) and MUS1, a DNA mismatch repair protein from maize ortologous to MSH2 from Arabidopsis, which is involved in the normal morphology and development (Hoffman et al., 2004).

Noteworthy, VRN2, a vernalization-related gene isolated from winter wheat, whose repression result in “devernalization” and flowering (Yong et al., 2003) is also present in Cluster 2.

Hormone-Related Genes

AUX1, SAUR and ARRO1 and XET are four auxin-related genes included in cluster 1. AUX1 is involved in auxin transport and accumulation (Kramer, 2004). SAUR, is an auxin-inducible gene. Constitutive expression to ARRO1 suggests that its role may be linked to the regulation of natural auxin levels within plant tissues (Butler and Gallagher, 2000). Cluster 2, characterised by a gradual decrease in expression, also contained several auxin-related genes such as TIR1, recently proposed as an auxin receptor (Kepinski and Leyser, 2005), PIN1 an auxin-efflux regulator involved in the directional flow (Friml et al., 2004) and one member to the SINA family, SINAH1, a component to the E3 ubiquitin ligase complex. Xie et al., (2002) reported SINAT5 promotes post-translational degradation of NAC1 (OsNAC6 is present in Cluster 1) to attenuate auxin signals. Auxin could be involved in the regulation of carbohydrate import to the spike in wheat (Darussalam et al., 1998). AIR3 is an auxin-induced protein involved in lateral root formation in Arabidopsis with characteristics of subtilisin-like proteases (Neuteboom et al., 1999).

More than 4% of the cluster 1 genes of known function are involved in ethylene synthesis/signaling or expressed during senescence. These genes include ethylene-forming-enzyme-like dioxygenase. Cluster 1 also contains the enzyme Allene Oxide Synthase (AOS), which is the first enzyme in the lipoxygenase (LOX) pathway involved in jasmonate biosynthesis (Maucher et al., 2000). Both ethylene and jasmonate are positive regulators of senescence, and could be involved in the degeneration of higher-order florets.

Genes Selectively Affected by Photoperiod

Several genes showed relatively stable expression under SD and increasing expression under LD, particularly during the second half of the experimental period (Cluster 3, FIG. 4). More than 30% of these genes have predicted function in signal transduction and more than 50% of these genes are kinases or kinase-like protein genes. APK2a, for instance is a serine/threonine protein kinase, involved in floral development (Ito et al., 1997). Other genes in this cluster include NAM (No Apical Meristem), a gene that determines the position of meristems and primordia (Souer et al., 1996), APETALA2, a MADS-box transcription factor involved in the control of flowering in Arabidopsis, and GA 20-oxidase, which is involved in the control of growth and flowering (Magome et al., 2004); CP-MII.3 (Serine carboxypeptidase II-3 precursor), a gibberellic acid-induced gene from barley involved in grain development (Dal Degan et al., 1994); SP1, a serine protease expressed in seeds and shoots of rice seedlings and immature siliques and flowers in Arabidopsis (Yamagata et al., 2000); GER7 a germin-like protein expressed during early phases of fiber development in cotton (Kim et al., 2004); TED2, which is expressed in immature primary xylem cells and in immature phloem cells (Demura and Fukuda, 1994); Aldehyde Oxidase (AO), which in maize is involved in the control of development (Sekimoto et al., 1997); and several genes with predicted function in pollen grains such as EXL3 (Mayfield et al., 2001), two Pollen-allergen protein, and Ascorbate Oxidase (Albani et al., 1992).

Several genes showed higher expression under LD over-imposed to a decreasing expression levels with development (Cluster 4). These genes include TIMING OF CAB EXPRESSION, which is a key component of the circadian clock also involved in light responses (Mas et al., 2003), PAP1 (IAA26) involved in control of transcriptional activity of primary auxin response genes (Reed, 2001; Tiwari, 2001) and JUBEL2 a homologue of BELL1 (Muller et al., 2001).

Other genes showed the opposite pattern, i.e. relatively stable expression under SD and decreasing expression under LD, particularly during the second half of the experiment (Cluster 6). Several of the genes of this group are involved in the control of fruit grown and development in other species. They include ERECTA (Shpak et al., 2004), ORFX/fw2.2 (Frary et al., 2000), and an SQUAMOSA-like gene (Muller et al., 2001).

Conclusions

The analysis of the transcriptome has revealed a relatively simple pattern of changes in gene expression. As little as six clusters are enough to include a very high proportion of the genes showing significant changes in expression during ear development between terminal spickelet stage and anthesis. The pattern of each one of these clusters is also relatively simple, dominated by either increasing or decreasing expression without large deviations from this general temporal trend. The robustness of the pattern is supported by the observation that functionally related genes converge to the same cluster.

Genes involved in the synthesis of the photosynthetic apparatus and photoprotection via anthocyanin screens as well as some and light perception genes increase their expression during the development of the ear inside the poor light environment created by the leaf sheath tube. For days, the gradual response of these genes anticipates the functional adjustment of the ear to the sudden transition to full light experienced during the emergence from the leaf cover. The increase in carbohydrate metabolism genes could either represent an anticipated adjustment to the grain filling stage starting after anthesis or a response to increased carbohydrate import rates. The steady rate of dry matter accumulation favours the first interpretation. Thus, while key developmental events are taking place between terminal spickelet and anthesis in terms of floret development, a large set of genes appears to prepare post-anthesis ear physiology. Whether the balance of resource investment in these two major avenues is optimised in terms of grain yield is not know.

Several genes, whose role in flower development and/or cell proliferation has been established by genetic analysis mainly in Arabidopsis, show decreasing expression levels between terminal spickelet and anthesis. It is tempting to speculate that genetic manipulation leading to sustained expression of these genes could reduce the observed abortion of florets, which limits grain yield in wheat.

Material and Methods

Plant Material

Plants of wheat (Triticum aestivum) cultivar Buck Manantial were sown (July, 2003 and July, 2004) at a density of 240 plants m⁻² at the experimental field of the Faculty of Agronomy, University of Buenos Aires (37°34′S, 58°20′W), on a silt clay loam classified as Aeric Argiudol (USDA taxonomy). Urea was added at sowing at a rate of 120 kg of nitrogen ha⁻¹. Weeds were controlled manually and fungicides and pesticides were applied to prevent fungal diseases and insect damage. Rainfall was complemented throughout the crop cycle by irrigation. Each replicate plot consisted of nine 1.2 m-long rows. Average temperature after terminal spickelet was 19° C.

(1) Photoperiod Treatments

Photoperiod treatments were applied only during the spike growth period, i.e. between terminal spikelet (30 September) initiation and anthesis. Plants were either exposed to the natural photoperiod of the growing season (approx. 12.5 h) or to natural photoperiods followed by a light extension of 6 h provided by a mixture of incandescent and fluorescent lamps. The photosynthetic photon flux density (400-700 nm) of the supplementary light was 4 μmol·m⁻²·s⁻¹ (measured on top of the canopy with a LI-COR Inc., Lincoln, Nebr., quantum sensor) and the red to far-red ratio 1.17 (measured with a SKR 110 660/730 sensor, Skye Instrument Ltd., Powys, UK). Thus, the extension made a negligible contribution to photosynthesis and did not significantly alter the natural red to far-red ratio.

(2) RNA Samples

The main shoot of field-grown plants was harvested on liquid nitrogen at midday. The spike was dissected under magnifying glass in a laboratory located by the field plots immediately after harvest. RNA was extracted from the main shoot spike by using NucleoSpin® RNA Plant (Macherey-Nagel). RNA quality was assessed by gel electrophoresis.

(a) Statistics

In each one of the experiments, a single microarray was used for each harvest time and photoperiod condition in combination with frequent sampling. This design favours a detailed description and a robust statistical analysis of the time course of expression of those genes showing gradual changes with time. The trade-off of this choice is that the design has no power to support statistically those genes showing peaks of expression restricted to a single harvest time but a preliminary inspection carried out before statistical analysis revealed no cluster with such feature.

For each experiment, normalised data were log-transformed and fitted to regression models including log harvest time (linear and quadratic terms) and either light treatment (SD or LD) or log harvest time×light treatment as independent variables. The p values were transformed into probabilities of false positives among selected genes (q, Storey and Tibshirani, 2003). All the genes presented in the text showed q values lower than 0.05 in each one of the two independent experiments. Thus, the probability of false positives among these genes is lower than 0.05.

Clusters (De Smet et al., 2002) are based on the first of the two field experiments. The probability of a gene to be included in a given cluster was set at 80% and minimum number of genes per cluster required was 5, because more stringent sorting resulted in large number of clusters with relatively small differences in pattern.

(b) Physiological Measurements

The dry weight of the spike was measured after drying for at least 3 days at 62° C., samples collected simultaneously with those used for the analysis of gene expression.

Thermal time was calculated as the sum of the differences between the daily mean air temperature minus the base temperature (0° C.).

### REFERENCES

Aarts, M. G. M., Keijzer, C. J., Stiekema, W. J. and Pereira, A. 1995. Molecular Characterization of the CER1 Gene of Arabidopsis Involved in Epicuticular Wax Biosynthesis and Pollen Fertility. Plant Cell 7, 2115-2127.
Aarts M G, Hodge R, Kalantidis K, Florack D, Wilson Z A, Mulligan B J, Stiekema W J, Scott R, Pereira A. 1997. The Arabidopsis MALE STERILITY 2 protein shares similarity with reductases in elongation/condensation complexes. Plant J. 12(3):615-23.
Albani D, Sardana R, Robert L S, Altosaar I, Arnison P G, Fabijanski S F. A Brassica napus gene family which shows sequence similarity to ascorbate oxidase is expressed in developing pollen. Molecular characterization and analysis of promoter activity in transgenic tobacco plants. 1992. Plant J. 2(3):331-42.
Albrecht V, Weinl S, Blazevic D, D'Angelo C, Batistic O, Kolukisaoglu U, Bock R, Schulz B, Harter K, Kudla J. The calcium sensor CBL1 integrates plant responses to abiotic stresses. 2003. Plant J. 36(4):457-70.
Wierzbicki A T, Jerzmanowski A. Suppression of Histone H1 Genes in Arabidopsis Results in Heritable Developmental Defects and Stochastic Changes in DNA Methylation. 2005. Genetics 169: 997-1008.
Aoyama K, Kawaura R, Yamada H, Aiba H, Mizuno T. Identification and characterization of a novel gene, hos3+, the function of which is necessary for growth under high osmotic stress in fission yeast. 2000. Biosci Biotechnol Biochem. 64(5):1099-102.
Armstrong S J, Caryl A P, Jones G H, Franklin F C. 2002. Asyl, a protein required for meiotic chromosome synapsis, localizes to axis-associated chromatin in Arabidopsis and Brassica. J Cell Sci. 15; 115(Pt 18):3645-55.
Bellmann R, Werr W. Zmhox1a, the product of a novel maize homeobox gene, interacts with the Shrunken 26 bp feedback control element. 1992. EMBO J. 11 (9):3367-74.
Bots M, Feron R, Uehlein N, Weterings K, Kaldenhoff R, Mariani T. PIP1 and PIP2 aquaporins are differentially expressed during tobacco anther and stigma development. 2005. J Exp Bot. 56(409):113-21.
Butler E D, Gallagher T F. Characterization of auxin-induced ARRO-1 expression in the primary root of Malus domestica. 2000. J Exp Bot. 51(351):1765-6.
Chen C N, Chu C C, Zentella R, Pan S M, Ho T H. AtHVA22 gene family in Arabidopsis: phylogenetic relationship, ABA and stress regulation, and tissue-specific expression. 2002. Plant Mol. Biol. 49(6):633-44.
Chen X, Liu J, Cheng Y, Jia D. HEN1 functions pleiotropically in Arabidopsis development and acts in C function in the flower. Development. 2002 March; 129(5): 1085-94.
Collinge M, Boller T. Differential induction of two potato genes, Stprx2 and StNAC, in response to infection by Phytophthora infestans and to wounding. 2001. Plant Mol. Biol. 46(5):521-9.
Conner J, Liu Z. LEUNIG, a putative transcriptional corepressor that regulates AGAMOUS expression during flower development. Proc Natl Acad Sci USA. 2000 Nov. 7; 97(23):12902-7.
Dal Degan F, Rocher A, Cameron-Mills V, von Wettstein D. The expression of serine carboxypeptidases during maturation and germination of the barley grain. 1994. Proc Natl Acad Sci USA. 16; 91(17):8209-13.
Darussalam C M, Patrick, J W. 1998. Auxin control of photoassimilate transport to and within developing grains of wheat within developing grains of wheat. Australian Journal of Plant Physiology 25, 69-77.
Davis S J, Bhoo S H, Durski A M, Walker J M, Vierstra R D. The heme-oxygenase family required for phytochrome chromophore biosynthesis is necessary for proper photomorphogenesis in higher plants. 2001. Plant Physiol. 126(2):656-69.
Demura T, Fukuda H. Novel vascular cell-specific genes whose expression is regulated temporally and spatially during vascular system development. 1994. Plant Cell. 6(7):967-81.
Ender F, Hallmann A, Amon P, Sumper M. Response to the sexual pheromone and wounding in the green alga volvox: induction of an extracellular glycoprotein consisting almost exclusively of hydroxyproline. 1999. J Biol. Chem. 3; 274(49):35023-8.
Fiebig A, Mayfield J A, Miley N L, Chau S, Fischer R L, Preuss D. Alterations in CER6, a gene identical to CUT1, differentially affect long-chain lipid content on the surface of pollen and stems. 2000. Plant Cell. 12(10):2001-8.
Frank De Smet, Janick Mathys, Kathleen Marchal, Gert Thijs, Bart De Moor and Yves Moreau. 2002. Adaptive Quality-based clustering of gene expression profiles. 2002. Bioinformatics, 18(6), 735-746.
Friml J, Yang X, Michniewicz M, Weijers D, Quint A, Tietz O, Benjamins R, Ouwerkerk P B, Ljung K, Sandberg G, Hooykaas P J, Palme K, Offring a R. A PINOID-dependent binary switch in apical-basal PIN polar targeting directs auxin efflux. Science. 2004 Oct. 29; 306(5697):862-5.
Gao M J, Parkin I, Lydiate D, Hannoufa A. An auxin-responsive SCARECROW-like transcriptional activator interacts with histone deacetylase. 2004. Plant Mol. Biol. May; 55(3):417-31.
Gebbing, T and Schnyder, H. 1999. Pre-Anthesis Reserve Utilization for Protein and Carbohydrate Synthesis in Grains of Wheat. Plant Physiology, 121, 871-878.
Gonzalez, F. G., Slafer, G. A., Miralles, D. J. 2005. Floret development and survival in wheat plants exposed to contrasting photoperiod and radiation environments during stem elongation. Functional Plant Biology 32, 189-197.
Gonzalez, F. G., Slafer, G. A., Miralles, D. J. 2003. Floret development and spike growth as affected by photoperiod during stem elongation in wheat. Field Crops Research 81, 29-38.
Green L S, Rogers E E. FRD3 controls iron localization in Arabidopsis. 2004. Plant Physiol. 136(1):2523-31.
Haga K, Takano M, Neumann R, Iino M. The Rice COLEOPTILE PHOTOTROPISM1 Gene Encoding an Ortholog of Arabidopsis NPH3 Is Required for Phototropism of Coleoptiles and Lateral Translocation of Auxin. Plant Cell. 2005 January; 17(1):103-15.
Hanaoka H, Noda T, Shirano Y, Kato T, Hayashi H, Shibata D, Tabata S, Ohsumi Y. Leaf senescence and starvation-induced chlorosis are accelerated by the disruption of an Arabidopsis autophagy gene. Plant Physiol. 2002 July; 129(3): 1181-93.
Hauser B A, He J Q, Park S O, Gasser C S. TSO1 is a novel protein that modulates cytokinesis and cell expansion in Arabidopsis. 2000 Development. 127(10):2219-26.
He J X, Gendron J M, Yang Y, Li J, Wang Z Y. 2002. The GSK3-like kinase BIN2 phosphorylates and destabilizes BZR1, a positive regulator of the brassinosteroid signaling pathway in Arabidopsis. Proc Natl Acad Sci USA 99: 10185-10190.
Hoffman P D, Leonard J M, Lindberg G E, Bollmann S R, Hays J B. Rapid accumulation of mutations during seed-to-seed propagation of mismatch-repair-defective Arabidopsis. 2004. Genes Dev. 1; 18(21):2676-85.
Hudson M E, Lisch D R, Quail P H. The FHY3 and FAR1 genes encode transposase-related proteins involved in regulation of gene expression by the phytochrome A-signaling pathway. Plant J. 2003 May; 34(4):453-71.
Ito T, Takahashi N, Shimura Y, Okada K. A serine/threonine protein kinase gene isolated by an in vivo binding procedure using the Arabidopsis floral homeotic gene product, AGAMOUS. Plant Cell Physiol. 1997 March; 38(3):248-58.
Jang C S, Lee M S, Kim J Y, Kim D S, Seo Y W. Molecular characterization of a cDNA encoding putative calcium binding protein, HvCaBP1, induced during kernel development in barley (Hordeum vulgare L.). 2003. Plant Cell Rep. 22(1):64-70.
Jansen R, Tollervey D, Hurt E C. A U3 snoRNP protein with homology to splicing factor PRP4 and G beta domains is required for ribosomal RNA processing. 1993. EMBO J. 12(6):2549-58.
Jeong J, Adamson L K, Hatam R, Greenhalgh D G, Cho K. Alterations in the expression and modifications of histones in the liver after injury. 2003. Exp. Mol. Path. 75:256-264.
Jwa N S, Kumar Agrawal G, Rakwal R, Park C H, Prasad Agrawal V. Molecular cloning and characterization of a novel Jasmonate inducible pathogenesis-related class 10 protein gene, JIOsPR10, from rice (Oryza sativa L.) seedling leaves. 2001. Biochem Biophys Res Commun. 7; 286(5):973-83.
Kang H G, Jang S, Chung J E, Cho Y G, An G. Characterization of two rice MADS box genes that control flowering time. 1997. Mol. Cells. 3; 7(4):559-66.

Karniol B, Chamovitz D A. The COP9 signalosome: from light signaling to general developmental regulation and back. 2000. Curr Opin Plant Biol. 3(5):387-93.
Kepinski S, Leyser O. 2005. The Arabidopsis F-box protein TIR1 is an auxin receptor. Nature 435, 446-51.
Kidner C A, Martienssen R A. The role of ARGONAUTE1 (AGO1) in meristem formation and identity. 2005. Dev Biol. 15; 280(2):504-17.
Kikuchi K, Ueguchi-Tanaka M, Yoshida K T, Nagato Y, Matsusoka M, Hirano H Y. Molecular analysis of the NAC gene family in rice. 2000. Mol Gen Genet. 262(6):1047-51.
Kim H J, Pesacreta T C, Triplett B A. Cotton-fiber germin-like protein. II: Immunolocalization, purification, and functional analysis. 2004. Planta. 218(4):525-35. Epub 2003 Nov. 21.
Kimura S, Ueda T, Hatanaka M, Takenouchi M, Hashimoto J, Sakaguchi K. Plant homologue of flap endonuclease-1: molecular cloning, characterization, and evidence of expression in meristematic tissues. 2000. Plant Mol. Biol. 42(3):415-27.
Kojima S, Takahashi Y, Kobayashi Y, Monna L, Sasaki T, Araki T, Yano M. Hd3a, a rice ortholog of the Arabidopsis FT gene, promotes transition to flowering downstream of Hd1 under short-day conditions. 2002. Plant Cell Physiol. 43(10): 1096-105.
Koning, A. J.; Tanimoto, E. Y.; Kiehne, K.; Rost, T.; Comai, L. Rockville, Md. Cell-specific expression of plant histone H2A genes. 1991. The Plant cell v. 7 (3): 657-665
Kosugi S, Ohashi Y. E2Ls, E2F-like repressors of Arabidopsis that bind to E2F sites in a monomeric form. 2002. J Biol. Chem. 10; 277(19):16553-8. Epub 2002 Feb. 26.
Kramer E M. PIN and AUX/LAX proteins: their role in auxin accumulation. 2004. Trends Plant Sci. 9(12):578-82.
Lai C P, Lee C L, Chen P H, Wu S H, Yang C C, Shaw J F. Molecular analyses of the Arabidopsis TUBBY-like protein gene family. 2004. Plant Physiol. 134(4): 1586-97.
Laxmi A, Paul L K, Peters J L, Khurana J P. Arabidopsis constitutive photomorphogenic mutant, bls1, displays altered brassinosteroid response and sugar sensitivity. 2004. Plant Mol. Biol. 56(2):185-201.
Lee S, Jeon J S, An K, Moon Y H, Lee S, Chung Y Y, An G. Alteration of floral organ identity in rice through ectopic expression of OsMADS16. 2003. Planta. 217(6):904-11.
Li J, Wang X Q, Watson M B, Assmann S M. Regulation of abscisic acid-induced stomatal closure and anion channels by guard cell AAPK kinase. Science. 2000 Jan. 14; 287(5451):300-3.
Lindsay W P, McAlister F M, Zhu Q, He X Z, Droge-Laser W, Hedrick S, Doemer P, Lamb C, Dixon R A. KAP-2, a protein that binds to the H-box in a bean chalcone synthase promoter, is a novel plant transcription factor with sequence identity to the large subunit of human Ku autoantigen. 2002. Plant Mol. Biol. 49(5):503-14.
Lindstrom, J. T., Lei, C.-H., Jones, M. L., Woodson, W. R. 1999. Accumulation of 1-aminocyclopropane-1-carboxylic acid (ACC) in Petunia pollen is associated with expression of a pollen-specific ACC synthase late in development. Journal of the American Society for Horticultural Science 124, 145-151.
Liscum E, Briggs W R. Mutations in the NPH1 locus of Arabidopsis disrupt the perception of phototropic stimuli. 1995. Plant Cell. 7(4):473-85.
Liu, Z., Running, M. P., Meyerowitz, E. M. 1997. TSO1 functions in cell division during Arabidopsis flower development. Development 124, 665
Lolle S J, Hsu W, Pruitt R E. Genetic analysis of organ fusion in Arabidopsis thaliana. 1998. Genetics. 149(2):607-19.
López-Fernández L A, López-Arañón D M, Castañeda V, Krimer D B, Del Mazo J. Developmental expresión of H3.3 variants histone mRNA in mouse. 1997. Int. J. Dev. Biol. 41:699-703.
Lynn K, Fernandez A, Aida M, Sedbrook J, Tasaka M, Masson P, Barton M K. The PINHEAD/ZWILLE gene acts pleiotropically in Arabidopsis development and has overlapping functions with the ARGONAUTE1 gene. 1999. Development. 126(3):469-81.
Macknight R, Bancroft I, Page T, Lister C, Schmidt R, Love K, Westphal L, Murphy G, Sherson S, Cobbett C, Dean C. FCA, a gene controlling flowering time in Arabidopsis, encodes a protein containing RNA-binding domains. Cell. 1997 May 30; 89(5):737-45.
Magome H, Yamaguchi S, Hanada A, Kamiya Y, Oda K. dwarf and delayed-flowering 1, a novel Arabidopsis mutant deficient in gibberellin biosynthesis because of overexpression of a putative AP2 transcription factor. 2004. Plant J. 37(5):720-9.
Mandaokar A, Kumar V D, Amway M, Browse J. Microarray and differential display identify genes involved in jasmonate-dependent anther development. Plant Mol. Biol. 2003 July; 52(4):775-86.
Mas P, Alabadi D, Yanovsky M J, Oyama T, Kay S A. 2003. Dual role of TOC1 in the control of circadian and photomorphogenic responses in Arabidopsis. Plant Cell 15, 223-36.
Maucher H, Hause B, Feussner I, Ziegler J, Wasternack C. Allene oxide synthases of barley (Hordeum vulgare cv. Salome): tissue specific regulation in seedling development. 2000. Plant J. 21(2):199-213.
Mayfield J A, Fiebig A, Johnstone S E, Preuss D. Gene families from the Arabidopsis thaliana pollen coat proteome. 2001. Science. 29; 292(5526):2482-5.
Miralles, D. J., Richards, R. A., Slafer, G. A. 2000. Duration of the stem elongation period influences the number of fertile florets in wheat and barley. Australian Journal of Plant Physiology 27, 931-940.
Monke G, Altschmied L, Tewes A, Reidt W, Mock H P, Baumlein H, Conrad U. Seed-specific transcription factors ABI3 and FUS3: molecular interaction with DNA. Planta. 2004 May; 219(1):158-66. Epub 2004 Feb. 7.
Morel, J. B., Godon, C., Mourrain, P., Béclin, C., Boutet, S., Feuerbach, F., Proux, F., Vaucheret, H. 2002. Fertile Hypomorphic ARGONAUTE (ago1) Mutants Impaired in Post-Transcriptional Gene Silencing and Virus Resistance. Plant Cell 14, 629-639
Muller B M, Saedler H, Zachgo S. The MADS-box gene DEFH28 from Antirrhinum is involved in the regulation of floral meristem identity and fruit development. Plant J. 2001 October; 28(2):169-79.
Muller J, Wang Y, Franzen R, Santi L, Salamini F, Rohde W. In vitro interactions between barley TALE homeodomain proteins suggest a role for protein-protein associations in the regulation of Knox gene function. 2001. Plant J. 27(1):13-23.
Naik, P. K., Mohapatra, P. K. Ethylene inhibitors promote male gametophyte survival in rice. 1999. Plant Growth Regulation 28, 29-39.
Neuteboom L W, Veth-Tello L M, Clijdesdale O R, Hooykaas P J, van der Zaal B J. A novel subtilisin-like protease gene from Arabidopsis thaliana is expressed at sites of lateral root emergence. 1999. DNA Res. 26; 6(1):13-9.
Nonomura K I, Nakano M, Murata K, Miyoshi K, Eiguchi M, Miyao A, Hirochika H, Kurata N. An insertional mutation in the rice PAIR2 gene, the ortholog of Arabidopsis ASY1, results in a defect in homologous chromosome pairing during meiosis. 2004. Mol Genet Genomics. 271(2):121-9.
Ohto M A, Fischer R L, Goldberg R B, Nakamura K, Harada J J. Control of seed mass by APETALA2. 2005. Proc Natl Acad Sci USA 102(8):3123-8.
Ohno R, Takumi S, Nakamura C. Kinetics of transcript and protein accumulation of a low-molecular-weight wheat LEA D-11 dehydrin in response to low temperature. 2003. J Plant Physiol. 160(2):193-200.
Pérez-Pérez José Manuel, Ponce María Rosa, and Micol José Luis. The ULTRACURVATA2 Gene of Arabidopsis Encodes an FK506-Binding Protein Involved in Auxin and Brassinosteroid Signaling. Plant Physiol. 2004 January; 134(1): 101-117.
Prigge, M. J., Otsuga, D., Alonso, J. M., Ecker, J. R., Drews, G. N., Clark, S. E. 2005. Class III homeodomain-leucine zipper gene family members have overlapping, antagonistic, and distinct roles in Arabidopsis development. Plant Cell 17, 61-76.
Prisco M, Maiorana A, Guerzoni C, Calin G, Calabretta B, Voit R, Grummt I, Baserga R. Role of pescadillo and upstream binding factor in the proliferation and differentiation of murine myeloid cells. 2004. Mol Cell Biol. 24(12):5421-33.
Prymakowska-Bosak M, Przew oka M R, Iusarczyk J, Kura M, Lichota J, Kilia czyk B, Jerzmanowski A. Linker Histones Play a Role in Male Meiosis and the Development of Pollen Grains in Tobacco. 1999. The Plant Cell, 11, 2317-2329,
Qiu Q S, Guo Y, Dietrich M A, Schumaker K S, Zhu J K Regulation of SOS1, a plasma membrane Na+/H+ exchanger in Arabidopsis thaliana, by SOS2 and SOS3. 2002. Proc Natl Acad Sci USA. 11; 99(12):8436-41.
Reed J W. Roles and activities of Aux/IAA proteins in Arabidopsis. 2001. Trends Plant Sci. 6(9):420-5.
Rogers E E, Guerinot M L. FRD3, a member of the multidrug and toxin efflux family, controls iron deficiency responses in Arabidopsis. 2002. Plant Cell. 14(8): 1787-99.
Sano R, Juarez C M, Hass B, Sakakibara K, Ito M, Banks J A, Hasebe M. KNOX homeobox genes potentially have similar function in both diploid unicellular and multicellular meristems, but not in haploid meristems. Evol Dev. 2005 January-February; 7(1):69-78.

Sato Y, Nishimura A, Ito M, Ashikari M, Hirano H Y, Matsuoka M. Auxin response factor family in rice. Genes Genet Syst. 2001 December; 76(6):373-80.
Schneider K, Mathur J, Boudonck K, Wells B, Dolan L, Roberts K. The ROOT HAIRLESS 1 gene encodes a nuclear protein required for root hair initiation in Arabidopsis. 1998. Genes Dev. 1; 12(13):2013-21.
Sekimoto H, Seo M, Dohmae N, Takio K, Kamiya Y, Koshiba T. Cloning and molecular characterization of plant aldehyde oxidase. 1997. J Biol. Chem. 13; 272(24): 15280-5.

Sentoku N, Kato H, Kitano H, Imai R. OsMADS22, an STMADS11-like MADS-box gene of rice, is expressed in non-vegetative tissues and its ectopic expression induces spikelet meristem indeterminacy. Mol Genet Genomics. 2005 March; 273(1):1-9.
Serino G, Deng X W. The COP9 signalosome: regulating plant development through the control of proteolysis. 2003. Annu Rev Plant Biol. 54:165-82.
Shpak E D, Berthiaume C T, Hill E J, Torii K U. Synergistic interaction of three ERECTA-family receptor-like kinases controls Arabidopsis organ growth and flower development by promoting cell proliferation. 2004. Development. 131(7):1491-501.
Soto M, Requena J M, Quijada L, Alonso C. Organization, transcription and regulation of the Leishmania infantum histone H3 genes. 1996. Biochem. J. 318, 813-819.
Souer E, van Houwelingen A, Kloos D, Mol J, Koes R. The no apical meristem gene of Petunia is required for pattern formation in embryos and flowers and is expressed at meristem and primordia boundaries. Cell. 1996 Apr. 19; 85(2):159-70.
Talbert, P. B., Adler, H. T., Parks, D. W., Comai, L. 1995. The REVOLUTA gene is necessary for apical meristem development and for limiting cell divisions in the leaves and stems of Arabidopsis thaliana. Development 121, 2723-2735
Tiwari S B, Wang X J, Hagen G, Guilfoyle T J. AUX/IAA proteins are active repressors, and their stability and activity are modulated by auxin. 2001. Plant Cell. 13(12):2800-22.
Subramaniam, K., Abbo, S. Ueng, P. P. 1996. Isolation of two differentially expressed wheat ACC synthase cDNAs and the characterization of one of their genes with root-predominant expression.: Plant Molecular Biology 31, 1009-1020.
Takada Y, Ito A, Ninomiya C, Kakizaki T, Takahata Y, Suzuki G, Hatakeyama K, Hinata K, Shiba H, Takayama S. Isogai A, Watanabe M. Characterization of expressed genes in the SLL2 region of self-compatible Arabidopsis thaliana. 2001. DNA Res. 31; 8(5):215-9.
Takumi S, Kosugi T, Murai K, Mori N, Nakamura C. Molecular cloning of three homoeologous cDNAs encoding orthologs of the maize KNOTTED1 homeobox protein from young spikes of hexaploid wheat. Gene. 2000 May 16; 249(1-2):171-81.
Van Mechelen J R, Schuurink R C, Smits M, Graner A, Douma A C, Sedee N J, Schmitt N F, Valk B E. 1999. Molecular characterization of two lipoxygenases from barley. Plant Mol. Biol. 39(6):1283-98.
Xie Q, Guo H S, Dallman G, Fang S, Weissman A M, Chua N H. SINAT5 promotes ubiquitin-related degradation of NAC1 to attenuate auxin signals. Nature. 2002 Sep. 12; 419(6903):167-70.
Xu W, Campbell P, Vargheese A K, Braam J. The Arabidopsis XET-related gene family: environmental and hormonal regulation of expression. 1996. Plant J. 9(6):879-89.
Yamagata H, Uesugi M, Saka K, Iwasaki T, Aizono Y. Molecular cloning and characterization of a cDNA and a gene for subtilisin-like serine proteases from rice (Oryza sativa L.) and Arabidopsis thaliana. 2000. Biosci Biotechnol Biochem. 64(9): 1947-57.
Yau, C. P., Wang, L., Yu, M., Zee, S. Y., Yip, W. K. 2004. Differential expression of three genes encoding an ethylene receptor in rice during development, and in response to indole-3-acetic acid and silver ions. Journal of Experimental Botany 55, 547-556.

Yong W D, Xu Y Y, Xu W Z, Wang X, Li N, Wu J S, Liang T B, Chong K, Xu Z H, Tan K H, Zhu Z Q. Vernalization-induced flowering in wheat is mediated by a lectin-like gene VER2. 2003. Planta. 217(2):261-70.

TABLE 1
SEQUENCES

>Erecta_Wheat TaCHIP__Ta005587_at TaTIGR_TC272622
CCACGCGTCCGCAAGAAGAAAAACTGGACTGGGAGGCTAGACTCCAGATT
GCTCTCGGCACTGCCCAAGGGCTGGCTTATCTTCACCATGACTGCAGCCC
ACGGATAATTCACAGGGATGTAAAATCGAAGAATATCCTCCTGGACAAGG
ACTATGTGGCGCACCTTGCTGACTTTGGCATTGCCAAGAGCGTGTGCATC
TCCAAGACCCACACGTCAACCTATGTGATGGGCACAATTGGCTACATTGA
CCCTGAGTATGCGCGCACATCCCGCCTGAACGAGAAATCTGATGTGTACA
GCTACGGCATCGTCTTGCTCGAGTTGCTGACCGGGAAGAAGCCAGTTGAC
AATGAGTGCAACCTCCATCACCTGATCCTATCCAAAGCCGCGGACAACAC
CGTGATGGAGATGGTCGACCCAGACATCACCGCCACATGCAAGGATCTCG
GCGAGGTCAAGAGGATGTTCCAGCTGGCGCTCCTCTGCAGCAAGCGGCAG
CCGTCCGACCGGCCGACGATGCACGACGTCGTGCACGTCCTCAGCTGCCT
GGTCTGCCCAGAAGCGCCCCCGAAGCCGGCGCAGCCGCCGGCGTCGCCCC
AGTCGTCCACGGCCCCGAGCTACGTGAACGAGTACGTCAGCCTGAGAAGC
GGCAGCGCCCTCTCCTGCGCCAACTCGTCGAGCGCGTCCGACGCGGAGCT
CTTCCTCAAGTTCGGCAAGGCGATATCGCAGAACACAGAGTAGACTGACC
TGAGCTGCCATGGTAGCATTTTCGC
>Erecta_Maize ZmTIGR16_TC299245
ACAGGCTGTTTGACCTGAACCTTGCGAATAACCACCTTGAAGGACCAATT
CCTGACAACCTAAGTTCATGTGTGAATCTCAATAGCTTCAATGCTTATGG
CAACAAGTTAAATGGAACCATTCCTCGTTCGCTGCGGAAACTTGAAAGCA
TGACCTATTTAAATCTTTCATCAAATTTCATAAGTGGCTCTATTCCTATT
GAGCTATCAAGGATTAACAATTTGGACACGTTGGACTTATCCTGTAACAT
GATGACGGGTCCAATTCCATCATCCATTGGCAACCTAGAGCATCTATTGA
GGCTTAACTTGAGCAAGAATGATCTAGTTGGATTCATCCCTGCGGAGTTT
GGTAATTTGAGAAGTGTCATGGAGATTGATTTATCCTATAATCATCTTGG
TGGTCTGATTCCTCAAGAACTTGGAATGCTGCAAAACCTGATGTTGCTAA
AACTGGAAAACAACAATATAACTGGCGATGTCTCTTCTCTGATGAACTGC
TTCAGCCTCAATATCTTAAATGTGTCATACAATAATTTGGCTGGTGCTGT
CCCTACTGACAACAACTTCACACGGTTTTCACATGACAGCTTTTTAGGTA
ATCCTGGACTCTGTGGATATTGGCTTGGTTCTTCATGTCGTTCCACTGGC
CACCGAGACAAACCGCCAATCTCAAAGGCTGCCATAATTGGTGTTGCTGT
GGGTGGACTTGTTATCCTCCTGATGATCTTAGTAGCTGTATGCAGGCCAC
ACCATCCACCTGCTTTTAAAGATGCCACTGTAAGCAAGCCAGTGAGCAAT
GGTCCACCCAAGCTGGTGATCCTTCATATGAACATGGCTCTTCATGTCTT
TGATGATATAATGAGGATGACTGAGAACTTGAGTGAGAAATACATCATTG
GATACGGGGCATCAAGTACAGTTTATAAATGTGTTCTAAAGAATTGCAAA
CCAGTGGCAATAAAAAAGCTGTATGCCCACTACCCTCAGAGCCTTAAGGA
ATTTGAAACTGAGCTCGAGACTGTTGGTAGCATCAAACACCGGAATCTAG
TCAGCCTTGCCAAGGGTACTCGTTGTCACCTGTTGGGAACCTCCTCTTTT
ATGATTATATGGAGAGTGGCAGCTTATGGGATGTTTTACATGAAGGCTCA
TCCAAGAAGAACAAACTTGACTGGGTGACTCGCCTACGGATCGCTCTTGG
TGCAGCTCAAGGCCTCGCTTACCTTCACCATGACTGCAGCCCACGAATAA
TTCACCGGGACGTAAAATCAAAGAATATACTCCTCGACAAAGATTATGAG
GCGCATCTTACAGACTTCGGCATCGCTAAGAGCTTATGTGTCTCGAAGAC
TCACACGTCAACCTACGTCATGGGCACTATTGGTTACATTGATCCTGAGT
ACGCCCGCACCTCCCGCCTCAACGAGAAGTCTGATGTCTACAGCTACGGC
ATCGTTCTGCTGGAGCTGCTGACCGGCAAGAAGCCAGTGGACAACGAGTG
CAATCTCCATCACTTGATCCTATCGAAGACGGCGAGCAACGAGGTCATGG
AGACGGTGGACCCCGACGTGGGAGACACCTGCAAGGACCTGGGCGAGGTG
AAGAAGCTGTTCCAGCTGGCGCTCCTCTGCACCAAGCGGCAGCCCTCGGA
CCGGCCGACGATGCACGAGGTGGTGCGCGTCCTTGACTGCCTGGTGAACC
CGGAGCCGCCGCCGCAGCCGCAGCAGCAGCAGCAGAAGGCGCACGCGCAC
CACCAGCTGCCGCCGCAGCCGTCGCCGCCGGCCTACGTCGACGAGTACGT
CAGCCTGCGGGGCACCGGCGCCCTCTCCTGCGCCAACTCGTCCAGCACCT
CGGACGCCGAGCTGTTCCTCAAGTTCGGCGAGGCCATCTCGCAGAACATG
GTGTAGGGGAAGACGTAGACGTTCGGTGAGGCGCCTTGAGTGAGGCCGAT
TGCAGGGGGAGTAGTTTGACTGACATTTTGTGGGACGCAGCGCAGGAGAT
TAACATGGGACTCAGTAGCTAGGGTGTTGTTAGCTGTAAAAAAAGTCATG
TGACGCAAGAGCAGCGGAGCTTCTTCCTCTTCTTTATCCCCCCTCCCCAT
TTTTCTTTGGTGGTCTAACTTACTAGGAGGCTGTATTGATCCATCATCAT
CTCTCCCGTTCCTCTGCTTGATGATCTTTTGTGACTTTCGCGGTCCTCTG
TATTGATCCATGATCTTTTGTG
>Erecta_genebank_maize AY106598.1
CCTGGACTCTGTGGATATTGGCTTGGTTCTTCATGTCGTTCCACTGGCCA
CCGAGACAAACCGCCAATCTCAAAGGCTGCCATAATTGGTGTTGCTGTGG
GTGGACTTGTTATCCTCCTGATGATCTTAGTAGCTGTATGCAGGCCACAC
CATCCACCTGCTTTTAAAGATGCCACTGTAAGCAAGCCAGTGAGCAATGG
TCCACCCAAGCTGGTGATCCTTCATATGAACATGGCTCTTCATGTCTTTG
ATGATATAATGAGGATGACTGAGAACTTGAGTGAGAAATACATCATTGGA
TACGGGGCATCAAGTACAGTTTATAAATGTGTTCTAAAGAATTGCAAACC
AGTGGCAATATTAAAAGCTGTATGCCCACTACCCTGCAGAGCCTTAAGGA
ATTTGAAACTGAGCTCGAGACTGTTGGTAGCATCAAACACCGGAATCTAG
TCAGCCTTGCCAAGGGTACTCGTTGTCACCTGTTGGGAACCTCCTCTTTT
ATGATTATATGGAGAGTGGCAGCTTATGGGATGTTTTACATGAAGGCTCA
TCCAAGAAGAACAAACTTGACTGGGTGACTCGCCTACGGATCGCTCTTGG
TGCAGCTCAAGGCCTCGCTTACCTTCACCATGACTGCAGCCCACGAATAA
TTCACCGGGACGTAAAATCAAAGAATATACTCCTCGACAAAGATTATGAG
GCCCATCTTACAGACTTCGGCATCGCTAAGAGCTTATGTGTCTCGAAGAC
TCACACGTCAACCTACGTCATGGGCACTATTGGTTACATTGATCCCGAGT
ACGCCCGCACCTCCCGCCTCAACGAGAAGTCTGATGTCTACAGCTACGGC
ATCGTTCTGCTGGAGCTGCTGACCGGCAAGAAGCCAGTGGACAACGAGTG
CAATCTCCATCACTTGATCCTATCGAAGACGGCGAGCAACGAGGTCATGG
AGACGGTGGACCCCGACGTGGGAGACACCTGCAAGGACCTGGGCGAGGTG
AAGAAGCTGTTCCAGCTGGCGCTCCTCTGCACCAAGCGGCAGCCCTCGGA
CCGGCCGACGATGCACGAGGTGGTGCGCGTCCTTGACTGCCTGGTGAACC
CGGAGCCGCCGCCGCAGCCGCAGCAGCAGCAGCAGAAGGGCGCACGCGCA
CCACCAGCTGCCGCCGCAGCCGTCGCCGCCGGCCTACGTCGACGAGTACG
TCAGCCTGCGGGGCACTGGCGCCCTCTCCTGCGCCAACTCGTCCAGCACC
TCGGACGCCGAGCTGTTCCTCAAG
>ORFx/fw2.2_Wheat TaCHIP_Ta007459_at
TaTIGR_TC253455
ACGCTTGACCTTAGGCCTATTTAGGTGACTTTTCAGCACAAGGTTGTACA
AAAAAGCAGGTTCCTACCGGTCCGGAATTCCCGGGATATCGTCGACCCAC
GCGTCCGCTCGTCCTTGCTCCCAAGCAATGTATCCCACCACCCCCTCCGA
CGCGTACGACAGGTTCAGCAACGGCCCTCCGCCCACGGCGCCGCCGCCGC
AGCAGCAGCCCACGTACAACCACGCCATGAACCAGAGCCACCACGCCCGC
CCCGCCGCCGGGCTGGCGCGCTGGTCCACCGGCCTCTTCCACTGCATGGA
CGACCCGGGAAACTGTCTCATCACGTGCCTGTGCCCCTGCATCACGTTCG
GGCAGATCGCGGACATCGTGGACAGAGGAACCTGCTCGTGCGCCGGGAGC
GGGGCGGCCTACGCGGCCATCTGCGCGACGACGGGGATGGGGTGCCTCTA
CTCGTGCGTCTACCGCACCAAGATGCGGGCGCACTACGACCTGGACGAGG
GGGAGTGCCCGGACTTCCTCGTGCATTGGTGCTGCGAGCTCTGCGCGCTC
TGCCAGGAGTACCGGGAGCTCAAGAACCGCGGCTTCGACATGGGGATCGG
CTGGGACGCCAACATGGAGCGCCGGAACCGGGGCGTCACCGGAGGGCAGG
TCATGGGGGCGCCGGCCACGCCCGTCGGCATGATGAGATAGGCAGGCCAC
GGCCGCGAAATCTCCGGTGATTTTCATGCGTCATGCTCCTCCCATCCCCT
GTGCCATTTCTCCTTGTGTTATTCTGGTTTGGAGATCGTTCGATTGACTG
GTTTTCTAGTAGCGTGCGCATTGTCTACTACTAGTTTGCTTGATGTGCCG
AATTATGTTGATTCTATGTAAAAATTTGTGGCGTGTTCGGCGTGTTC
>ORFx/fw2.2_Maize ZmTIGR16_DN228992
GCGCGTTGCTGTTCGCATAAACAAGAGATCCTAGTTCTACTCCCAAGCAA
TGTATCCGTCAGCCCCTCCGGACGCGTATAACAAGTTCAGCGCCGGGGCT
CCGCCAACGGCGCCGCCGCCGCCGGCAGCGTACCAGCAGCAGCAGCACGG
GGCGAACATGGACACTTCACGCCCCGGCGGCGGGCTGAGGAAATGGTCCA
CCGGCCTCTTCCACTGCATGGACGACCCGGGGAACTGTCTCATCACATGC
CTGTGCCCCTGTGTCACGTTCGGGCAGGTCGCTGACATCGTGGACAAGGG
CACCTGCCCATGTATCGCGAGCGGACTGGTGTACGGGCTCATCTGCGCGT
CGACGGGGATGGGGTGCCTGTACTCGTGCCTCTACCGGTCCAAGCTGAGG
GCCGAGTACGACCTGGACGAAGGGGAGTGCCCGGACATCCTGGTGCACTG
CTGCTGCGAGCACCTGGCTCTGTGCCAGGAGTACCGCGAGCTCAAGAACC
GCGGCTTCGACCTGGGGATCGGCTGGGAGGCTAACATGGACCGGCAGAGA
CGAGGAGTTGCCGGCGGCGGC
>Squamosa_like_Wheat TaCHIP_Ta002552_at
TaTIGR_TC233332
CTCCTTCTCCTTCCACTCATCAGCTAGCTCCACAGTTCCACTTCCGCGAC
AGCTCCCAGAAAAGAGCCCTAACTAGCTTCATCTTCATCGCGCCTCACCG
GAAGGCATGGACCGCAAGGACAAGTCCCGCAAGTCCTCCTCGGCAGCGTC
CATGGCCGCGCTCGCCGCCGCAGCTGCGGCCGGCGACGTGGCCCGAGCCG
ACGGGATGTCCGGCGAGGAGGACCAGAAGCTGAAGCTCGTGAACGTTCCC
GTGGTCTCCGTCGGCGGCTCGAGCTCCTCTGCCGCGGCGGTGGCGGTGAG
GAGGGGCAGTGGTGCTGCTGGCGCCGTGGCGACGGGCGCGGCTGGGGCAG
GCGGGCCGAGCTGCCAGGCTGAGAGGTGCGGCGCCGACCTCAGCGAGGCG
AAGCGGTACCACCGCAGGCACAAGGTGTGCGAGGCGCACTCCAAGGCTGC
TGTCGTGGTCGTCGCCGGCCTCCGCCAGCGCTTCTGCCAGCAATGCAGCC
GGTTCCACGAGCTTTTGGAGTTCGACGACCAGAAGCGCAGCTGCCGCCGG
CGCCTGGCCGGGCACAACGAGCGGCGGAGGAAGAGCTCGGCGGAGGCCAA
CGGCGGCGACGGGTGCCGCCACGCCGACCAGGACGGCCGTAGCCACCCGG
GGAACCCGCCGCTGAACCATTTCCAGATCAGATAAACCTACCTAAGCATG
CTCCCTCTCTTCTGTCATGCAGCCATGCTACTAGCTACATATGTATCCAC
CGACCTATGTACGTACCTGCCTACCTACGTCTCCACTATCCGGTGTACTA
ACTGGCAGTATATGTGGTCTCCTGTACTGCTATATATTGCTGCTGTCTGC
CAAAAACCTAAGCTAAGCTAATTGTAACCTGGGAACTGGTCAGGTGTTAG
AGTGATTGTGTGTGTGATGTGTAATTGTATGGCCTGTGCAGCCGAGACTT
CTGCAGGCTGTGTAATATTGCTGCTGTGCTGGGATAATTTGTGGCAATAT
ATGCTATCTATTCGAGT
>Squamosa_like_Maize ZmTIGR16_TC305152
GCTTTAAATCCGCCTCCTCCCAGCGTCTCCCTCCTCCCGGCCTGTCCCCT
CACCTCCCTCCCCCTATCTCTCCACCGCCGCAGCTAGCTGCGACGTCATG
CACTCGCCGGCGCCACCGCCACCGCATACTATCTACAATTAGCCAGCCGT
AGGCTTACCTATCCTGTGTCAAGCAAGCCTCTCGCAAGCAACAAGGAAGG
AAGCTAGCTAGTTTTATAGCTGCTGTCGGCGGCGGCGGCTGAAGCGACGT
GCCTGAGCTAGGATTTAGGTTGAGATCAGGAGAGGGAGAAGGCGGCGGCG
ATGGGGCGCGGGAAGGTGCAGCTGAAGCGGATCGAGAACAAGATCAACCG
CCAGGTGACATTCTCCAAGCGCCGCTCGGGGCTACTCAAGAAGGCGCACG
AGATCTCCGTGCTCTGCGACGCCGAGGTCGCGCTCATCATCTTCTCCACC
AAGGGCTAAGCTCTACGAGTACTCTACCGATTCATGTATGGACAAAATTC
TTGAACGGTATGAGCGCTACTCCTATGCAGAAAAGGTTCTCATTTCCGCA
GAATATGAAACTCAGGGCTAATTGGTGCCATGAATATAGAAAACTAAAGG
CGAAGGTCGAGACAATACAGAAATGTCAAAAGCACCTCATGGGAGAGGAT
CTTGAAACTTTGAATCTCAAAGAGCTTCAGCAACTAGAGCAGCAGCTGGA
GAGTTCACTGAAACATATCAGAACAAGGAAGAGCCAGCTTATGGTCGAGT
CAATTTCAGCGCTCCAACGGAAGGAGAAGTCACTGCAGGAGGAGAACAAG
GTTCTGCAGAAGGAGCTCGCGGAGAAGCAGAAAGACCAGCGGCAGCAAGT
GCAACGGGACCAAACTCAACAGCAGACCAGTTCGTCTTCCACGTCCTTCA
TGTTAAGGGAAGCTGCCCCAACAACAAATGTCAGCATCTTCCCTGTGGCA
GCAGGCGGGAGGGTGGTGGAAGGGGCAGCAGCGCAGCCGCAGGCTCGCGT
TGGACTGCCACCATGGATGCTTAGCCATCTGAGCTGCTGAAGGTTTGCCC
TCACCGTCGCGATGTGATAGCCCAAAATCCAGCAAGTCTTCCCCGGACCC
CGGACTGTCCCCGCTTCTCCGCCTGGTGCAGTATCGTCGTGATCGCGAGA
AGCAGCAGCAGCGGTTTGCCGTATTTTTTACCACTGTGTATAAGTAGCAT
ATCTGCAATGTGTATATTTTGTTCACCGTTCCTGCTGGGGCGGCCGCACG
GTACTTTACGCATGTAATATATGTGCTTCATCAAATCACTTTCACTACCA
TAGCATTTGCCGGTTTGTGGTAGTTTCATGTCTCACATAATGGGTACACA
TTCATATCCCAGTGGTCTCGGTTCTTC
>Squamosa_like_Maize GB:AJ430641.1 Zea mays mRNA
for putative MADS-domain transcription factor
(m4 gene)
ATGGGGCGCGGGAAGGTGCAGCTGAAGCGGATCGAGAACAAGATCAACCG
CCAGGTGACATTCTCCAAGCGCCGCTCGGGGCTACTCAAGAAGGCGCACG
AGATCTCCGTGCTCTGCGACGCCGAGGTCGCGCTCATCATCTTCTCCACC
AAGGGCAAGCTCTACGAGTACTCTACCGATTCATGTATGGACAAAATTCT
TGAACGGTATGAGCGCTACTCCTATGCAGAAAAGGTTCTCATTTCCGCAG
AATATGAAACTCAGGGCTAATTGGTGCCATGAATATAGAAAACTAAAGGC
GAAGGTCGAGACAATACAGAAATGTCAAAAGCACCTCATGGGAGAGGATC
TTGAAACTTTGAATCTCAAAGAGCTTCAGCAACTAGAGCAGCAGCTGGAG
AGTTCACTGAAACATATCAGAACAAGGAAGAGCCAGCTTATGGTCGAGTC
AATTTCAGCGCTCCAACGGAAGGAGAAGTCACTGCAGGAGGAGAACAAGG
TTCTGCAGAAGGAGCTCGCGGAGAAGCAGAAAGACCAGCGGCAGCAAGTG
CAACGGGACCAAACTCAACAGCAGACCAGTTCGTCTTCCACGTCCTTCAT
GTTAAGGGAAGCTGCCCCAACAACAAATGTCAGCATCTTCCCTGTGGCAG
CAGGCGGGAGGGTGGTGGAAGGGGCAGCAGCGCAGCCGCAGGCTCGCGTT
GGACTGCCACCATGGATGCTTAGCCATCTGAGCTGCTGA
>BRI1_interactor_like_Wheat TaCHIP_Ta031245_at
TaTIGR_TC235401
AAAAGGGTGGCCTCGAAGTCATAGAAACTTGTGAGATATCCAGTAATGAA
GTTGTGGCTTCGGCCAAGACAATTATCACTTAAGCAACAAAAGAAGAAGC
ATAAGAAAACCAAAAAGCGTGTTAAGGACACGGCCAGTGTTGATTGTTCT
GGTGACAACATCAGTGAGCAGCAAACATCAACATCAGCAACTGTGCCGCC
CAAAACAGCAGAATCTACATCTCGTAAACGGAAATATTCCCATGCCAGTG
TAAGTTCTGAAAACAACACCCAAGCGCTCAAAGACAGGCAGCAGGCTGTT
GGAGCAAGTAGTGCTGGTGCCGGTGATGATAACATGAACACCAGGAACAG
AAAGAGTGGAACTTCTGCTAGCGCGGAGCCTCCATAAGTTGGGTTCAATC
TCTTCTACAGATCAAGCACATTCAAGAAACAACGTAGATGCAAAGAGGGG
AGCCGCCTCCCAACAAGTCGTTATTCGCACAGAGGACTTGAGGGCTGAAG
TTACTGTTCCATGCTGGGATGATGTTGCCGTGCCAAGCAGTGAGACTAGA
GAGTACAAAAGTTCAGGGAAGAAGAGCATTGGTTATGTGCTGGATGAGTG
GGATGAGGAGTACGACCGTGGAAAGGCAAAGAAGGTCAGGAAAGCGAGGG
AAGACGACGACGATGACGGCATGAACCCCTTCCAGGCGGAGGCCAACTAC
GTGTCAGCGCGTAAGACGAAACAGAATTCCTACAAATCCAAGCCTTGGGA
CAAGCCCGCCAGACCGAGTCGCTAGACTTTGCTGAAAGAGATGGCGCCCC
GAGGCTCCGTAGAAAGAGAAGAGAAGTGTTGTTGTATTGCCAATTTTTCG
AAATATACCGTCTGCTGTTCCTTTTTTTCCCGCCTTGTTGCTCCTTTTTT
TCACCTTGTTGTTTGACTAGACGGCTCGATTAGACAGAACTCTTGATCGG
GGCCTGTATTTAATTGTGAGATTCTTTGTAGCGCAGCCGGAGTAGTAGAA
CATGTTGCCGTTTTTCGCAGCATTTTTCGTTCTTGTTTCTCCAGCAACAT
AGTTATGATTTGTTATGAATAAACATTTGTTCTCGTTTCTTGGCGGTAC
>BRI1_interactor_like_Maize ZmTIGR16_TC299392
GACGGCGGCCAGAAAAAAGAAAAAGAAAAAAAAAACAGCTCGCCGCACCA
ATCTCCCCTACCATATCGATCGACCCTGCTAGCTTTAATCCAACAGAATT
CCACCGGAAATTTGGTGAAGTGCTAATCGCTTGCGGCTTGCCTGCCCGGT
CTCGTGGCTTTGTGCTGCTCCCGCTGTGGTGAGGAACCCTAGGATGGCGG
GGGTTTCGATGGCGGCGGCGCCTGAGGGGGTGCTGCACCGCCGGATTGAG
TTTCATCTTGCACGGAGACCGCATGGCTCTGTGGCGGTGGGCGGCGGCGG
GTTCCGGATGGAGACGCTGAACCCGGACGCCGCTGGTAAGGTGGGGCCCG
GGGAGGCGGTCGGGAGCAGCGAGGGGGATGCGAGGAGGCCGGAGAAGGGA
GACAGTGGCGGGATCGATCCGGAGCTCAGCGTCGCAAGGATCTACCTCGG
GAGAATTGGTGCTGGATTGGAAAATCTTGGGAATACCTGCTATCTCAACT
CAGTTCTGCAATGCTTGACGTACACGGAGCCGTTCGCTGCCTATTTGCAG
AGCGGGAAGCACACGTCCTCATGTCGCACATCCGGATTTTGTGCATTATG
TGCTCTTCAGATACATGTCAAGACTGCTCTAGAATCAACTGGAAAAACAG
TGACACCATTTCGTATTGTCAAGAACTTGCGCTGCATTTCTCGCACTTTC
CGAAATTCAAGACAAGAAGATGCACACGAGTTAATGGTTAATTTGCTTGA
ATCCATGCATAAATGCTGTTTACCTTCTGGGGTATCGACTGAGTCTCAAA
GTGCTTATGACAAAAGCTTAGTTCACAAAATATTTGGTGGCCGCCTTAGA
AAACAGGTCAAATGCACACGTTGCTTACATTGCTCCAATAAATTTGATCC
TTTCTTGGATCTCAGTCTTGAGATTGCCAAGGCCACGACTTTGGTGCGGG
CACTTGATAAATTTTACCGAGGATGAGCTACTAGACGAGGGTCAAAAGGA
ATACGAGTGTGAACGCTGCAGGCAGAAAGTTGTAGCAAAGAAACGACTTA
CAATTGATAAAGCCCCCAATGTCTTGACAATTCATCTGAAGCGCTTCAGC
CCTTTCAATCCTCGTGAGAAGATTGACAAAAAAGTGGACTTCCAATCAAT
TCTTGATTTGAAACCATTTGTTAGTGACTCTAAAGGAACAGATTTTAAAT
ACAGCCTTTATGGTGTCCTAGTCCATACAGGTTGGAGCACTCAATCAGGC
CATTACTATTGCTACGTTCGTACATCCAGCGGGATGTGGCACAATCTTGA
TGATAAAGAGGTGCGCCAAGTTCGTGAAGCAGACGTATTGAAGCAGAAGG
CTTATATGTTAUTTATGTACGTAACAGTATTGGGAAGTCGGTGGTGTGCA
AAGATAATAATGCTGCTAATTTGCTCAAGAAGAAAACCTCTGAAATAAAT
CTCAAGTTTGAATGGTATTGCACAAACCAATGTAAGAGCACAAAATTTGA
ATGGTGTGTCTCCATTTGGTGATAAGACGCACGGCACAATCATTGGCTAC
TCTACAATTTCCAGCAAGACTAAAACAATCCACTGTTCAAAGAATGAGGT
CAAAGCTGAAGATGCTCCAGCCTCTCAAAACAATGCTCTACCTTCCACAC
AAGCCCCCATAGCTCGAAATGATGTTGGTATCTTGCCAACTAAACCAATG
CAGTTTGCTGTAAAAAATCAGGAGAAAACCTCATCCCACCAACCTGCGCC
ATTTACAAATACTTGTGGTGAACAAACAGTTGTTGGGAGGCCCTTGCAGG
AAATGGAACCTAAGGCTGGTGCAGGGGAAAATACTTCTGTTGTCTCTGCT
GTTGCTAATGATGCCGCTACATTGTCCAAGGCAGACAAACTAACTTCTCA
GCCTCAAACAGCTCCATCTTCCAAGCCAACTGCCAATGTAAAGGACACAG
CTACTGAGTTTGCTACACTGTCTTTATCAAAGAAGGATTCTGTTGTTTCA
AATGGTGCTGTGCTTAGCATTGGATGTCTCACATCCGGTGAGAAAGCAAT
GAACTTATCAGAATCTGTGGATCAAGCTAATGAAATCACAAAGGCACTTC
CTACGAGTCAGAACAATACTGCACCAGTAATTGCTCAAGCAGATTGTGGA
GTAGAGATCAGCTCTGGTGTCATCATGCACGTTGCTGTGTCTGCTTCATG
CAATGGAACTACAGAGAAAAAGGTGAATTCGAAGTCAAAGAAGTTTGTGA
GGTATCCTGTTGTGAACATGTGGTTAGGGTCCAGACATCTAAAGCCAGGG
AAGAAAGCGAACCGCAAGAGAAGTAAAATGATAAGACGTGTTGTAGCCTG
CAAGGACGCGGCAAATTTATCTTGTTTGAGTGAGCAGCTGACGTCAACAT
CGTCCACTGCATGCTCTGAAACCGTAGAATGTACCTCCAGCCATCAGAAA
CGGTCACATGCTAGTGCAAGGTCCAAAGATGACGCCCAGTCCTCGCAAAA
CAAGCAGAAGGTCGACGGAGCTCGTGTCGGTGCTGGCACAAGTGCACCTT
CTTGCAGTGCCGACGTTCCAAAGTCTGATCCGAACCCTTCCACAGATGCT
AAGGTTGTAGCGACCCAGCCTGCCAGTATTCGCGCAACGGACCTGATGGA
AGCTACCGTTCCGCGCTGGGATGACGTAGACATGCCAAACATAAAGGTCG
CTGAGCCGCAGTCCAAACGCAAGAGCGGTGGCTACATCTTAGATGAGTGG
GACGAGGAGTACGACCGAGGGAAGACGAAGAAGGTGCGGAGGTCAAAGCA
AGACTTTGGCGGGCCAAACCCGTTCCAGGAGGAGGCGGACTACGTATCGC
AGCGGAGGACGAAGCAGAAATCCTACCGAGGCAAGTCTTGGAACAAGCGG
GCCTAACACGGTAGAGAGGGGGGAGTTGAGAGAATACGAGAGAGAGAGAG
AGAGAGAGAGAATCCCCGGACGCACATGCTGTTTGTTGTTACGTTTTAGC
ACCCATTGGTGAGGGAGATTTACGTTTAAAAACCATATCCGTCGTCGGTA
CGGTAGTCCCCATTAGTTGTCGACGATGTGACGTTTTGGGCAGTTTTCGT
AGAACATTTGAAACATTTGTTCGGTTGTTAACGGAGTGCTGTTTAGTTGA
GTTGGGGGGAACAAAATTGGCGAGACTATTTTGCTGCATTCTGCTCTGCT
GTTTTTAGTTACTGATGAAGAATAAAACGATGGTGGTGATCAACTCGTGC

Although the present invention has been described in some detail by way of illustration and example for purposes of clarity and understanding, it will be clear to those of skill in the art that certain changes and modifications may be practiced within the scope of the appended claims.

What is claimed is:

1. A method for improving the yield of a plant, the method comprising introducing into the plant an expression cassette capable of altering the expression in the plant of a gene selected from the group consisting of Erecta, ORFx/fw2.2, a squamosa-like gene, an m4 gene, and a BRI1 interactor-like gene, the nucleotide sequences of said genes set forth in Table 1, such that their relative level of expression of the gene in long days or short days is increased or decreased in relation to a wild-type plant, so that the overall yield of the plant at harvest is increased.

2. The method of claim 1 wherein the expression cassette is introduced into the plant using an Agrobacterium-mediated transformation method.

3. The method of claim 1 wherein the expression cassette is introduced into the plant using a microparticle bombardment-mediated transformation method.

4. The method of claim 1 wherein the expression cassette is introduced using a whiskers-based transformation method.

5. The method of claim 1 wherein the plant is selected from the group consisting of maize, cotton, soybean, canola, sunflower, wheat, buckwheat and alfalfa.

6. The method of claim 1 wherein the alteration of expression results in over-expression of the gene.

7. The method of claim 1 wherein the alteration of expression results in decreased expression of the gene.

8. The method of claim 3 wherein the decreased expression of the gene is accomplished through the use of a technology selected from the group consisting of antisense, co-suppression, and RNAi.


SEQUENCE LISTING (10 sequences)

SEQ ID NO: 1 - DNA, 775 bp, Triticum aestivum
ccacgcgtccgcaagaagaaaaactggactgggaggctagactccagattgctctcggcactgcccaagggctggcttatcttcaccatgactgcagcccacggataattcacagggatgtaaaatcgaagaatatcctcctggacaaggactatgtggcgcaccttgctgactttggcattgccaagagcgtgtgcatctccaagacccacacgtcaacctatgtgatgggcacaattggctacattgaccctgagtatgcgcgcacatcccgcctgaacgagaaatctgatgtgtacagctacggcatcgtcttgctcgagttgctgaccgggaagaagccagttgacaatgagtgcaacctccatcacctgatcctatccaaagccgcggacaacaccgtgatggagatggtcgacccagacatcaccgccacatgcaaggatctcggcgaggtcaagaggatgttccagctggcgctcctctgcagcaagcggcagccgtccgaccggccgacgatgcacgacgtcgtgcacgtcctcagctgcctggtctgcccagaagcgcccccgaagccggcgcagccgccggcgtcgccccagtcgtccacggccccgagctacgtgaacgagtacgtcagcctgagaagcggcagcgccctctcctgcgccaactcgtcgagcgcgtccgacgcggagctcttcctcaagttcggcaaggcgatatcgcagaacacagagtagactgacctgagctgccatggtagcattttcgc

SEQ ID NO: 2 - DNA, 2222 bp, Zea mays
acaggctgtttgacctgaaccttgcgaataaccaccttgaaggaccaattcctgacaacctaagttcatgtgtgaatctcaatagcttcaatgcttatggcaacaagttaaatggaaccattcctcgttcgctgcggaaacttgaaagcatgacctatttaaatctttcatcaaatttcataagtggctctattcctattgagctatcaaggattaacaatttggacacgttggacttatcctgtaacatgatgacgggtccaattccatcatccattggcaacctagagcatctattgaggcttaacttgagcaagaatgatctagttggattcatccctgcggagtttggtaatttgagaagtgtcatggagattgatttatcctataatcatcttggtggtctgattcctcaagaacttggaatgctgcaaaacctgatgttgctaaaactggaaaacaacaatataactggcgatgtctcttctctgatgaactgcttcagcctcaatatcttaaatgtgtcatacaataatttggctggtgctgtccctactgacaacaacttcacacggttttcacatgacagctttttaggtaatcctggactctgtggatattggcttggttcttcatgtcgttccactggccaccgagacaaaccgccaatctcaaaggctgccataattggtgttgctgtgggtggacttgttatcctcctgatgatcttagtagctgtatgcaggccacaccatccacctgcttttaaagatgccactgtaagcaagccagtgagcaatggtccacccaagctggtgatccttcatatgaacatggctcttcatgtctttgatgatataatgaggatgactgagaacttgagtgagaaatacatcattggatacggggcatcaagtacagtttataaatgtgttctaaagaattgcaaaccagtggcaataaaaaagctgtatgcccactaccctcagagccttaaggaatttgaaactgagctcgagactgttggtagcatcaaacaccggaatctagtcagccttgccaagggtactcgttgtcacctgttgggaacctcctcttttatgattatatggagagtggcagcttatgggatgttttacatgaaggctcatccaagaagaacaaacttgactgggtgactcgcctacggatcgctcttggtgcagctcaaggcctcgcttaccttcaccatgactgcagcccacgaataattcaccgggacgtaaaatcaaagaatatactcctcgacaaagattatgaggcgcatcttacagacttcggcatcgctaagagcttatgtgtctcgaagactcacacgtcaacctacgtcatgggcactattggttacattgatcctgagtacgcccgcacctcccgcctcaacgagaagtctgatgtctacagctacggcatcgttctgctggagctgctgaccggcaagaagccagtggacaacgagtgcaatctccatcacttgatcctatcgaagacggcgagcaacgaggtcatggagacggtggaccccgacgtgggagacacctgcaaggacctgggcgaggtgaagaagctgttccagctggcgctcctctgcaccaagcggcagccctcggaccggccgacgatgcacgaggtggtgcgcgtccttgactgcctggtgaacccggagccgccgccgcagccgcagcagcagcagcagaaggcgcacgcgcaccaccagctgccgccgcagccgtcgccgccggcctacgtcgacgagtacgtcagcctgcggggcaccggcgccctctcctgcgccaactcgtccagcacctcggacgccgagctgttcctcaagttcggcgaggccatctcgcagaacatggtgtaggggaagacgtagacgttcggtgaggcgccttgagtgaggccgattgcagggggagtagtttgactgacattttgtgggacgcagcgcaggagattaacatgggactcagtagctagggtgttgttagctgtaaaaaaagtcatgtgacgcaagagcagcggagcttcttcctcttctttatcccccctccccatttttctttggtggtctaacttactaggaggctgtattgatccatcatcatctctcccgttcctctgcttgatgatcttttgtgactttcgcggtcctctgtattgatccatgatcttttgtg

SEQ ID NO: 3 - DNA, 1273 bp, Zea mays
cctggactctgtggatattggcttggttcttcatgtcgttccactggccaccgagacaaaccgccaatctcaaaggctgccataattggtgttgctgtgggtggacttgttatcctcctgatgatcttagtagctgtatgcaggccacaccatccacctgcttttaaagatgccactgtaagcaagccagtgagcaatggtccacccaagctggtgatccttcatatgaacatggctcttcatgtctttgatgatataatgaggatgactgagaacttgagtgagaaatacatcattggatacggggcatcaagtacagtttataaatgtgttctaaagaattgcaaaccagtggcaataaaaaagctgtatgcccactaccctgcagagccttaaggaatttgaaactgagctcgagactgttggtagcatcaaacaccggaatctagtcagccttgccaagggtactcgttgtcacctgttgggaacctcctcttttatgattatatggagagtggcagcttatgggatgttttacatgaaggctcatccaagaagaacaaacttgactgggtgactcgcctacggatcgctcttggtgcagctcaaggcctcgcttaccttcaccatgactgcagcccacgaataattcaccgggacgtaaaatcaaagaatatactcctcgacaaagattatgaggcccatcttacagacttcggcatcgctaagagcttatgtgtctcgaagactcacacgtcaacctacgtcatgggcactattggttacattgatcccgagtacgcccgcacctcccgcctcaacgagaagtctgatgtctacagctacggcatcgttctgctggagctgctgaccggcaagaagccagtggacaacgagtgcaatctccatcacttgatcctatcgaagacggcgagcaacgaggtcatggagacggtggaccccgacgtgggagacacctgcaaggacctgggcgaggtgaagaagctgttccagctggcgctcctctgcaccaagcggcagccctcggaccggccgacgatgcacgaggtggtgcgcgtccttgactgcctggtgaacccggagccgccgccgcagccgcagcagcagcagcagaagggcgcacgcgcaccaccagctgccgccgcagccgtcgccgccggcctacgtcgacgagtacgtcagcctgcggggcactggcgccctctcctgcgccaactcgtccagcacctcggacgccgagctgttcctcaag

SEQ ID NO: 4 - DNA, 897 bp, Triticum aestivum
acgcttgaccttaggcctatttaggtgacttttcagcacaaggttgtacaaaaaagcaggttcctaccggtccggaattcccgggatatcgtcgacccacgcgtccgctcgtccttgctcccaagcaatgtatcccaccaccccctccgacgcgtacgacaggttcagcaacggccctccgcccacggcgccgccgccgcagcagcagcccacgtacaaccacgccatgaaccagagccaccacgcccgccccgccgccgggctggcgcgctggtccaccggcctcttccactgcatggacgacccgggaaactgtctcatcacgtgcctgtgcccctgcatcacgttcgggcagatcgcggacatcgtggacagaggaacctgctcgtgcgccgggagcggggcggcctacgcggccatctgcgcgacgacggggatggggtgcctctactcgtgcgtctaccgcaccaagatgcgggcgcactacgacctggacgagggggagtgcccggacttcctcgtgcattggtgctgcgagctctgcgcgctctgccaggagtaccgggagctcaagaaccgcggcttcgacatggggatcggctgggacgccaacatggagcgccggaaccggggcgtcaccggagggcaggtcatgggggcgccggccacgcccgtcggcatgatgagataggcaggccacggccgcgaaatctccggtgattttcatgcgtcatgctcctcccatcccctgtgccatttctccttgtgttattctggtttggagatcgttcgattgactggttttctagtagcgtgcgcattgtctactactagtttgcttgatgtgccgaattatgttgattctatgtaaaaatttgtggcgtgttcggcgtgttc

SEQ ID NO: 5 - DNA, 571 bp, Zea mays
gcgcgttgctgttcgcataaacaagagatcctagttctactcccaagcaatgtatccgtcagcccctccggacgcgtataacaagttcagcgccggggctccgccaacggcgccgccgccgccggcagcgtaccagcagcagcagcacggggcgaacatggacacttcacgccccggcggcgggctgaggaaatggtccaccggcctcttccactgcatggacgacccggggaactgtctcatcacatgcctgtgcccctgtgtcacgttcgggcaggtcgctgacatcgtggacaagggcacctgcccatgtatcgcgagcggactggtgtacgggctcatctgcgcgtcgacggggatggggtgcctgtactcgtgcctctaccggtccaagctgagggccgagtacgacctggacgaaggggagtgcccggacatcctggtgcactgctgctgcgagcacctggctctgtgccaggagtaccgcgagctcaagaaccgcggcttcgacctggggatcggctgggaggctaacatggaccggcagagacgaggagttgccggcggcggc

SEQ ID NO: 6 - DNA, 1017 bp, Triticum aestivum
ctccttctccttccactcatcagctagctccacagttccacttccgcgacagctcccagaaaagagccctaactagcttcatcttcatcgcgcctcaccggaaggcatggaccgcaaggacaagtcccgcaagtcctcctcggcagcgtccatggccgcgctcgccgccgcagctgcggccggcgacgtggcccgagccgacgggatgtccggcgaggaggaccagaagctgaagctcgtgaacgttcccgtggtctccgtcggcggctcgagctcctctgccgcggcggtggcggtgaggaggggcagtggtgctgctggcgccgtggcgacgggcgcggctggggcaggcgggccgagctgccaggctgagaggtgcggcgccgacctcagcgaggcgaagcggtaccaccgcaggcacaaggtgtgcgaggcgcactccaaggctgctgtcgtggtcgtcgccggcctccgccagcgcttctgccagcaatgcagccggttccacgagcttttggagttcgacgaccagaagcgcagctgccgccggcgcctggccgggcacaacgagcggcggaggaagagctcggcggaggccaacggcggcgacgggtgccgccacgccgaccaggacggccgtagccacccggggaacccgccgctgaaccatttccagatcagataaacctacctaagcatgctccctctcttctgtcatgcagccatgctactagctacatatgtatccaccgacctatgtacgtacctgcctacctacgtctccactatccggtgtactaactggcagtatatgtggtctcctgtactgctatatattgctgctgtctgccaaaaacctaagctaagctaattgtaacctgggaactggtcaggtgttagagtgattgtgtgtgtgatgtgtaattgtatggcctgtgcagccgagacttctgcaggctgtgtaatattgctgctgtgctgggataatttgtggcaatatatgctatctattcgagt

SEQ ID NO: 7 - DNA, 1375 bp, Zea mays
gctttaaatccgcctcctcccagcgtctccctcctcccggcctgtcccctcacctccctccccctatctctccaccgccgcagctagctgcgacgtcatgcactcgccggcgccaccgccaccgcatactatctacaattagccagccgtaggcttacctatcctgtgtcaagcaagcctctcgcaagcaacaaggaaggaagctagctagttttatagctgctgtcggcggcggcggctgaagcgacgtgcctgagctaggatttaggttgagatcaggagagggagaaggcggcggcgatggggcgcgggaaggtgcagctgaagcggatcgagaacaagatcaaccgccaggtgacattctccaagcgccgctcggggctactcaagaaggcgcacgagatctccgtgctctgcgacgccgaggtcgcgctcatcatcttctccaccaagggcaagctctacgagtactctaccgattcatgtatggacaaaattcttgaacggtatgagcgctactcctatgcagaaaaggttctcatttccgcagaatatgaaactcagggcaattggtgccatgaatatagaaaactaaaggcgaaggtcgagacaatacagaaatgtcaaaagcacctcatgggagaggatcttgaaactttgaatctcaaagagcttcagcaactagagcagcagctggagagttcactgaaacatatcagaacaaggaagagccagcttatggtcgagtcaatttcagcgctccaacggaaggagaagtcactgcaggaggagaacaaggttctgcagaaggagctcgcggagaagcagaaagaccagcggcagcaagtgcaacgggaccaaactcaacagcagaccagttcgtcttccacgtccttcatgttaagggaagctgccccaacaacaaatgtcagcatcttccctgtggcagcaggcgggagggtggtggaaggggcagcagcgcagccgcaggctcgcgttggactgccaccatggatgcttagccatctgagctgctgaaggtttgccctcaccgtcgcgatgtgatagcccaaaatccagcaagtcttccccggaccccggactgtccccgcttctccgcctggtgcagtatcgtcgtgatcgcgagaagcagcagcagcggtttgccgtattttttaccactgtgtataagtagcatatctgcaatgtgtatattttgttcaccgttcctgctggggcggccgcacggtactttacgcatgtaatatatgtgcttcatcaaatcactttcactaccatagcatttgccggtttgtggtagtttcatgtctcacataatgggtacacattcatatcccagtggtctcggttcttc

SEQ ID NO: 8 - DNA, 738 bp, Zea mays
atggggcgcgggaaggtgcagctgaagcggatcgagaacaagatcaaccgccaggtgacattctccaagcgccgctcggggctactcaagaaggcgcacgagatctccgtgctctgcgacgccgaggtcgcgctcatcatcttctccaccaagggcaagctctacgagtactctaccgattcatgtatggacaaaattcttgaacggtatgagcgctactcctatgcagaaaaggttctcatttccgcagaatatgaaactcagggcaattggtgccatgaatatagaaaactaaaggcgaaggtcgagacaatacagaaatgtcaaaagcacctcatgggagaggatcttgaaactttgaatctcaaagagcttcagcaactagagcagcagctggagagttcactgaaacatatcagaacaaggaagagccagcttatggtcgagtcaatttcagcgctccaacggaaggagaagtcactgcaggaggagaacaaggttctgcagaaggagctcgcggagaagcagaaagaccagcggcagcaagtgcaacgggaccaaactcaacagcagaccagttcgtcttccacgtccttcatgttaagggaagctgccccaacaacaaatgtcagcatcttccctgtggcagcaggcgggagggtggtggaaggggcagcagcgcagccgcaggctcgcgttggactgccaccatggatgcttagccatctgagctgctga

SEQ ID NO: 9 - DNA, 1100 bp, Triticum aestivum
aaaagggtggcctcgaagtcatagaaacttgtgagatatccagtaatgaagttgtggcttcggccaagacaattatcacttaagcaacaaaagaagaagcataagaaaaccaaaaagcgtgttaaggacacggccagtgttgattgttctggtgacaacatcagtgagcagcaaacatcaacatcagcaactgtgccgcccaaaacagcagaatctacatctcgtaaacggaaatattcccatgccagtgtaagttctgaaaacaacacccaagcgctcaaagacaggcagcaggctgttggagcaagtagtgctggtgccggtgatgataacatgaacaccaggaacagaaagagtggaacttctgctagcgcggagcctccaaagttgggttcaatctcttctacagatcaagcacattcaagaaacaacgtagatgcaaagaggggagccgcctcccaacaagtcgttattcgcacagaggacttgagggctgaagttactgttccatgctgggatgatgttgccgtgccaagcagtgagactagagagtacaaaagttcagggaagaagagcattggttatgtgctggatgagtgggatgaggagtacgaccgtggaaaggcaaagaaggtcaggaaagcgagggaagacgacgacgatgacggcatgaaccccttccaggcggaggccaactacgtgtcagcgcgtaagacgaaacagaattcctacaaatccaagccttgggacaagcccgccagaccgagtcgctagactttgctgaaagagatggcgccccgaggctccgtagaaagagaagagaagtgttgttgtattgccaatttttcgaaatataccgtctgctgttcctttttttcccgccttgttgctcctttttttcaccttgttgtttgactagacggctcgaaatagacagaactcttgatcggggcctgtatttaattgtgagattctttgtagcgcagccggagtagtagaacatgttgccgtttttcgcagcatttttcgttcttgtttctccagcaacatagttatgatttgttatgaataaacatttgttctcgtttctttggcggtac

SEQ ID NO: 10 - DNA, 3248 bp, Zea mays
gacggcggccagaaaaaagaaaaagaaaaaaaaaacagctcgccgcaccaatctcccctaccatatcgatcgaccctgctagctttaatccaacagaattccaccggaaatttggtgaagtgctaatcgcttgcggcttgcctgcccggtctcgtggctttgtgctgctcccgctgtggtgaggaaccctaggatggcgggggtttcgatggcggcggcgcctgagggggtgctgcaccgccggattgagtttcatcttgcacggagaccgcatggctctgtggcggtgggcggcggcgggttccggatggagacgctgaacccggacgccgctggtaaggtggggcccggggaggcggtcgggagcagcgagggggatgcgaggaggccggagaagggagacagtggcgggatcgatccggagctcagcgtcgcaaggatctacctcgggagaattggtgctggattggaaaatcttgggaatacctgctatctcaactcagttctgcaatgcttgacgtacacggagccgttcgctgcctatttgcagagcgggaagcacacgtcctcatgtcgcacatccggattttgtgcattatgtgctcttcagatacatgtcaagactgctctagaatcaactggaaaaacagtgacaccatttcgtattgtcaagaacttgcgctgcatttctcgcactttccgaaattcaagacaagaagatgcacacgagttaatggttaatttgcttgaatccatgcataaatgctgtttaccttctggggtatcgactgagtctcaaagtgcttatgacaaaagcttagttcacaaaatatttggtggccgccttagaaaacaggtcaaatgcacacgttgcttacattgctccaataaatttgatcctttcttggatctcagtcttgagattgccaaggccacgactttggtgcgggcacttgaaaattttaccgaggatgagctactagacgagggtcaaaaggaatacgagtgtgaacgctgcaggcagaaagttgtagcaaagaaacgacttacaattgataaagcccccaatgtcttgacaattcatctgaagcgcttcagccctttcaatcctcgtgagaagattgacaaaaaagtggacttccaatcaattcttgatttgaaaccatttgttagtgactctaaaggaacagattttaaatacagcctttatggtgtcctagtccatacaggttggagcactcaatcaggccattactattgctacgttcgtacatccagcgggatgtggcacaatcttgatgataaagaggtgcgccaagttcgtgaagcagacgtattgaagcagaaggcttatatgttattttatgtacgtaacagtattgggaagtcggtggtgtgcaaagataataatgctgctaatttgctcaagaagaaaacctctgaaaaaatctcaagtttgaatggtattgcacaaaccaatgtaagagcacaaaatttgaatggtgtgtctccatttggtgataagacgcacggcacaatcattggctactctacaatttccagcaagactaaaacaatccactgttcaaagaatgaggtcaaagctgaagatgctccagcctctcaaaacaatgctctaccttccacacaagcccccatagctcgaaatgatgttggtatcttgccaactaaaccaatgcagtttgctgtaaaaaatcaggagaaaacctcatcccaccaacctgcgccatttacaaatacttgtggtgaacaaacagttgttgggaggcccttgcaggaaatggaacctaaggctggtgcaggggaaaatacttctgttgtctctgctgttgctaatgatgccgctacattgtccaaggcagacaaactaacttctcagcctcaaacagctccatcttccaagccaactgccaatgtaaaggacacagctactgagtttgctacactgtctttatcaaagaaggattctgttgtttcaaatggtgctgtgcttagcattggatgtctcacatccggtgagaaagcaatgaacttatcagaatctgtggatcaagctaatgaaatcacaaaggcacttcctacgagtcagaacaatactgcaccagtaattgctcaagcagattgtggagtagagatcagctctggtgtcatcatgcacgttgctgtgtctgcttcatgcaatggaactacagagaaaaaggtgaattcgaagtcaaagaagtttgtgaggtatcctgttgtgaacatgtggttagggtccagacatctaaagccagggaagaaagcgaaccgcaagagaagtaaaatgataagacgtgttgtagcctgcaaggacgcggcaaatttatcttgtttgagtgagcagctgacgtcaacatcgtccactgcatgctctgaaaccgtagaatgtacctccagccatcagaaacggtcacatgctagtgcaaggtccaaagatgacgcccagtcctcgcaaaacaagcagaaggtcgacggagctcgtgtcggtgctggcacaagtgcaccttcttgcagtgccgacgttccaaagtctgatccgaacccttccacagatgctaaggttgtagcgacccagcctgccagtattcgcgcaacggacctgatggaagctaccgttccgcgctgggatgacgtagacatgccaaacataaaggtcgctgagccgcagtccaaacgcaagagcggtggctacatcttagatgagtgggacgaggagtacgaccgagggaagacgaagaaggtgcggaggtcaaagcaagactttggcgggccaaacccgttccaggaggaggcggactacgtatcgcagcggaggacgaagcagaaatcctaccgaggcaagtcttggaacaagcgggcctaacacggtagagaggggggagttgagagaatacgagagagagagagagagagagagaatccccggacgcacatgctgtttgttgttacgttttagcacccattggtgagggagatttacgtttaaaaaccatatccgtcgtcggtacggtagtccccattagttgtcgacgatgtgacgttttgggcagttttcgtagaacatttgaaacatttgttcggttgtaacggagtgctgtttagttgagttggggggaacaaaattggcgagactattttgctgcattctgctctgctgtttttagttactgatgaagaataaaacgatggtggtgatcaactcgtgc